(19) United States
(12) Patent Application Publication
(10) Pub. No.: US 2026/0275085 A1
(43) Pub. Date: Sep. 17, 2026

(54) METHODS FOR MANUFACTURING BIOMATERIAL-BASED MATERIALS VIA EXTRUSION

(71) Applicant: THE FYNDER GROUP, INC., Chicago, IL (US)

(72) Inventors: Christopher STASZEL, Chicago, IL (US); Eric EPSTEIN, Chicago, IL (US); Jeremy J. WEIGAND, Chicago, IL (US)

(21) Appl. No.: 19/469,240

(22) PCT Filed: Mar. 28, 2024
(86) PCT No.: PCT/US2024/022000

§ 371 Date: Sep. 25, 2025

Related U.S. Application Data
(60) Provisional application No. 63/455,238, filed on Mar. 28, 2023.
(60) Provisional application No. 63/519,349, filed on Aug. 14, 2023.

(51) Int. Cl. C08K 11/00; C08G 63/88; C08G 69/46; C08J 3/20

(52) U.S. Cl. CPC C08K 11/00; C08G 63/88; C08G 69/46; C08J 3/20

(57) ABSTRACT

Fibrous materials that include biomaterials are disclosed, as are methods for making and using such fibrous materials. In some embodiments, a biomaterial-based fibrous material is formed by extruding a deformable mixture comprising the biomaterial into a desired spatial configuration and subsequently treating the deformable mixture to form the fibrous material. In some cases, the fibrous material may be a textile and/or the biomaterial may comprise fungal biomass. The biomaterial-based fibrous materials disclosed herein may have advantageous mechanical and aesthetic properties that make the fibrous materials especially suitable for use, by way of non-limiting example, as leather analog materials.

### CROSS-REFERENCE TO RELATED APPLICATIONS

This application claims the benefit of priority of U.S. Provisional Patent Applications 63/455,238, filed 28 Mar. 2023; and 63/519,349, filed 14 Aug. 2023. Each of the above-referenced applications is incorporated herein by reference in its entirety.

### FIELD

This disclosure relates generally to biomaterial-based materials, such as textiles, and methods for the manufacture thereof, and particularly to methods of making biomaterial-based (e.g., fungal biomass-based) materials by the extrusion of materials comprising biomass (e.g., fungal biomass) or other biomaterials.

### BACKGROUND

Many current fibrous materials, including but not limited to textiles such as leather, create environmental problems during manufacturing and may be difficult or impossible to recycle or dispose of in an environmentally safe way at the end of an article's useful life. By way of non-limiting example, the manufacture of leather depends on the rearing of cattle (which has a significant environmental impact in itself and often raises animal welfare concerns) and requires a tanning step, which may use highly toxic chemicals such as chromium, formic acid, mercury, and various solvents. Leather also biodegrades slowly, over times of about 25 to 40 years. Many textile materials suffer from similar environmental or ethical concerns.

There is thus a need in the art for fibrous or similar materials, such as textiles, that may be produced cost-effectively with a minimum of environmental impact and without animal welfare or other ethical concerns. It is further advantageous for such materials to be imparted with or engineered to have various selected physical and/or mechanical properties, e.g., tensile strength, tear strength, flexural rigidity, elasticity, texture, thermal properties, sensory attributes, etc., of conventional fibrous materials, e.g., leather.

### SUMMARY

In an aspect of the present disclosure, a method for producing a biomaterial-based material comprises extruding a deformable mixture comprising a liquid fraction and a first biomaterial into a desired spatial configuration; and treating the extruded deformable mixture to form the biomaterial-based material.

In embodiments, the biomaterial-based material may be a textile.

In embodiments, the treating step may comprise at least one of removing at least a portion of the liquid fraction from the extruded deformable mixture, heating the extruded deformable mixture, cooling the extruded deformable mixture, and causing a chemical reaction in the extruded deformable mixture. The treating step may, but need not, comprise causing a chemical reaction in the extruded deformable mixture that results in curing or hardening of the extruded deformable mixture.

In embodiments, the extruding step may comprise at least one of single-screw and/or twin-screw extrusion. In embodiments, the extruding step may further comprise dual extrusion, co-extrusion, multiple head extrusion, and/or combinations thereof.

In embodiments, the deformable mixture may be pelletized in the extruding step and the method further comprises processing pellets of the pelletized deformable mixture by molding or a second extruding step.

In embodiments, the liquid fraction may make up no more than about 90 wt %, no more than about 85 wt %, no more than about 80 wt %, no more than about 75 wt %, no more than about 70 wt %, no more than about 65 wt %, no more than about 60 wt %, no more than about 55 wt %, no more than about 50 wt %, no more than about 45 wt %, no more than about 40 wt %, no more than about 35 wt %, no more than about 30 wt %, no more than about 25 wt %, no more than about 20 wt %, or no more than about 15 wt % of the deformable mixture.

In embodiments, the first biomaterial may make up at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, or at least about 85 wt % of the deformable mixture. Additionally or alternatively, the first biomaterial may make up less than about 85 wt %, less than about 80 wt %, less than about 75 wt %, less than about 70 wt %, less than about 65 wt %, less than about 60 wt %, less than about 55 wt %, less than about 50 wt %, less than about 45 wt %, less than about 40 wt %, less than about 35 wt %, less than about 30 wt %, less than about 25 wt %, less than about 20 wt %, less than about 15 wt %, or less than about 10 wt % of the deformable mixture.

In embodiments, the method may further comprise co-extruding a material that does not comprise the first biomaterial. The co-extruding step may, but need not, comprise co-extruding the deformable mixture with a coating or backing material. The co-extruding step may, but need not, comprise extruding a layer of the deformable mixture between first and second layers of the material that does not comprise the first biomaterial. The material that does not comprise the first biomaterial may, but need not, comprise a first polymer. The material that does not comprise the first biomaterial may, but need not, comprise a blend of polylactic acid (PLA) and a second thermoplastic. The second thermoplastic may, but need not, be a polyester. The second thermoplastic may, but need not, be a plasticized starch. The material that does not comprise the first biomaterial may, but need not, comprise a thermoplastic elastomer. The thermoplastic elastomer may, but need not, be a segmented polyester. The thermoplastic elastomer may, but need not, be a polyamide block copolymer. The material that does not comprise the first biomaterial may, but need not, further comprise one or more plasticizers that reduces the Young's modulus and/or flexural modulus of the polymer, one or more plasticizers that reduce the Young's modulus and/or flexural modulus of the material, one or more crosslinkers that crosslink the polymer, one or more crosslinkers that crosslink the material, or a combination thereof.

In embodiments, at least one of the deformable mixture and the material that does not comprise the first biomaterial may comprise at least one additive selected from the group consisting of pigments, foaming agents, crosslinkers, plasticizers, polymers other than the first polymer, and biomaterials other than the first biomaterial.

In embodiments, at least one of the following may be true: (i) in the extruding step, the deformable mixture is extruded into a first number of layers, wherein the first number of layers is an integer greater than or equal to 1 and less than or equal to 10,000; and (ii) in the co-extruding step, the material that does not comprise the first biomaterial is extruded into a second number of layers, wherein the second number of layers is an integer greater than or equal to 1 and less than or equal to 10,000.

In embodiments, the deformable mixture may be a foam and further comprise at least one gas. The at least one gas may, but need not, be selected from the group consisting of air, carbon dioxide, nitrogen, gaseous hydrocarbons (e.g., methane, ethane, propane, butane, etc.) and combinations thereof.

In embodiments, the extruding step may be a reactive extrusion step comprising simultaneously extruding the deformable mixture and chemically reacting the deformable mixture with a chemically reactive species. The chemically reactive species may, but need not, comprise at least one of an acrylate and an epoxy. The biomaterial may, but need not, have been functionalized with a crosslinking agent prior to the extruding step.

In embodiments, the deformable mixture may be a colloid in which the biomaterial is dispersed in the liquid fraction.

In embodiments, the deformable mixture may comprise one or more thermoplastic polymers. The one or more thermoplastic polymers may, but need not, comprise a plasticized thermoplastic, a thermoplastic elastomer, or a combination thereof. The one or more thermoplastic polymers may, but need not, comprise a polyester. The one or more thermoplastic polymers may, but need not, comprise an ethylene-vinyl acetate or ethylene-vinyl alcohol copolymer. The one or more thermoplastic polymers may, but need not, comprise a plasticized starch. The one or more thermoplastic polymers may, but need not, comprise a segmented polyester. The thermoplastic elastomer may, but need not, be a polyamide block copolymer. The liquid fraction may, but need not, comprise a non-aqueous thermoplastic and the biomaterial may, but need not, comprise dried fungal biomass. The dried fungal biomass may, but need not, have been dried by at least one of spray-drying, flash-drying, supercritical drying, and/or freeze-drying. The dried fungal biomass may have been sized-reduced to form a plurality of particles. The dried fungal biomass may, but need not, have been size-reduced by at least one of grinding, milling, cutting, and/or blending. In embodiments, the D₅₀ of the particles of fungal biomass may be about 17 μm to about 23 μm, about 18 μm to about 22 μm, about 19 μm to about 21 μm, and/or about 20 μm, and/or the D₉₀ of the particles of fungal biomass may be about 30 μm to about 40 μm, about 31 μm to about 39 μm, about 32 μm to about 38 μm, about 33 μm to about 37 μm, about 34 μm to about 36 μm, and/or about 35 μm.

In embodiments, the particle size distribution of the particles may be multimodal i.e., bimodal, trimodal, or consisting of four or more modes.

The method may, but need not, further comprise a thermoplastic processing step. The thermoplastic processing step may, but need not, comprise at least one of calendering, injection molding, and compression molding.

In embodiments, the thermoplastic polymer may make up about make up at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, or at least about 75 wt % of the deformable mixture. Additionally or alternatively, the thermoplastic polymer may make up less than about 75 wt %, less than about 70 wt %, less than about 65 wt %, less than about 60 wt %, less than about 55 wt %, less than about 50 wt %, less than about 45 wt %, less than about 40 wt %, less than about 35 wt %, less than about 30 wt %, or less than about 25 wt % of the deformable mixture.

In embodiments, the deformable mixture may further comprise a filler. The filler may, but need not, be selected from the group consisting of microfibrillated cellulose, nanofibrillated cellulose, recycled fibers, recycled particles, polymeric fibers, flame retardants, colored pigments, polymeric particles, and combinations thereof.

In embodiments, the desired spatial configuration may be a sheet and the extruding step comprises extruding the deformable mixture onto a substantially planar surface. The substantially planar surface may, but need not, be a textured surface. The substantially planar surface may, but need not, comprise biomass particles and be configured to release at least a portion of the biomass particles onto a surface of the extruded deformable mixture, such that the biomass particles thereby become embedded in the extruded deformable mixture, during or after the treating step.

In embodiments, the liquid fraction may be selected from the group consisting of water, one or more alcohols, and combinations thereof.

In embodiments, the deformable mixture may be a Bingham plastic.

In embodiments, the deformable mixture may further comprise a gelling agent or non-gelling polysaccharide. The gelling agent may, but need not, comprise a polymer having a molecular weight of at least about 80,000 daltons. The polymer may, but need not, be a polysaccharide, a polypeptide, a protein, a starch, a block copolymer, a polyelectrolyte, or a vegetable gum. The polymer may, but need not, be a hydrocolloid selected from the group consisting of t-karrageenan, κ-carrageenan, λ-carrageenan, agar, starch, modified starch, xanthan, guar gum, locust bean gum, gum arabic, acacia gum, gum karaya, gum tragacanth, alginate, pectin, methyl cellulose, hydroxypropylmethyl cellulose, carboxymethyl cellulose, and combinations thereof.

In embodiments, the deformable mixture may further comprise at least one plasticizer. The at least one plasticizer may, but need not, make up about 10 wt % to about 85 wt % of a total solids content of the deformable mixture. The at least one plasticizer may, but need not, comprise at least one of glycerol and urea.

In embodiments, the treating step may comprise removing at least a portion of the liquid fraction and is carried out by heating the deformable mixture, applying a negative pressure to the deformable mixture, radiofrequency irradiation of the deformable mixture, microwave irradiation of the deformable mixture, or a combination thereof.

In embodiments, the treating step may comprise removing at least a portion of the liquid fraction and a rate at which the at least a portion of the liquid fraction is removed during the treating step may be controlled, optimized, selected, or tuned to provide a preselected porosity to the biomaterial-based material.

In embodiments, the extruding step may comprise extruding the deformable mixture onto a surface of a backing material. The backing material may be free of adhesive.

In embodiment, the method may further comprise applying a topcoat to the biomaterial-based material. The topcoat may comprise polyurethanes, acrylics, isocyanates, silicone-based handfeel modifiers, dulling agents, wetting agents, pigments, and/or combinations thereof. The topcoating may be applied by at least one of transfer films, spray coating, and/or roll coating.

In embodiments, the method may further comprise embossing or imprinting a pattern or texture into a surface of the biomaterial-based material.

In embodiments, the biomaterial-based material may be recycled and re-extruded.

In another aspect of the present disclosure, a biomaterial-based material is made by any of the methods described herein.

In embodiments, at least one of the following may be true: (i) the first biomaterial makes up at least about 50%, at least about 60%, at least about 70%, at least about 75%, at least about 76%, at least about 77%, at least about 78%, at least about 79%, or at least about 80% by weight of the deformable mixture; (ii) the first biomaterial makes up at least about 10% by weight of the biomaterial-based material; (iii) a tensile strength of the biomaterial-based material is at least about 3 MPa, at least about 4 MPa, at least about 5 MPa, at least about 6 MPa, at least about 7 MPa, at least about 8 MPa, at least about 9 MPa, or at least about 10 MPa; and (iv) an elastic modulus of the biomaterial-based material is no more than about 70 MPa. Any two of (i), (ii), (iii), and (iv) may, but need not, be true. Any three of (i), (ii), (iii), and (iv) may, but need not, be true. All four of (i), (ii), (iii), and (iv) may, but need not, be true.

In embodiments, the biomaterial-based material may have a tensile strength of at least about 3 MPa. The biomaterial-based material may, but need not, have a tensile strength of at least about 10 MPa.

In embodiments, the biomaterial-based material may comprise at least one additive selected from the group consisting of fillers, functionalizing compounds, crosslinkers, polymers, sizing agents, hydrophobing agents, plasticizers, pigments, dyes, antifoaming agents, defoaming agents, flocculants, deflocculants, antimicrobial agents, antistatic agents, UV stabilizers, surface modifiers, foaming agents, blowing agents, and flame retardants. The at least one additive may, but need not, comprise a surface modifier selected from the group consisting of a texture modifier, a slip agent, a non-slip agent, a matting agent, and a gloss agent.

In embodiments, the biomaterial-based material may comprise two or more material layers. Each of the two or more material layers may, but need not, comprise the biomaterial. At least one of the two or more material layers may, but need not, be free of the biomaterial. The biomaterial-based material may, but need not, comprise ten or more material layers. At least one of the material layers may, but need not, have a thickness of no more than about 10 μm.

In embodiments, the biomaterial-based material may further comprise of a topcoat. The topcoat comprises polyurethanes, acrylics, isocyanates, silicone-based handfeel modifiers, dulling agents, wetting agents, pigments, and/or combinations thereof.

In embodiments, the biomaterial-based material may be embossed or imprinted with a pattern or texture into a surface of the biomaterial-based material.

While specific embodiments and applications have been illustrated and described, the present disclosure is not limited to the precise configuration and components described herein. Various modifications, changes, and variations which will be apparent to those skilled in the art may be made in the arrangement, operation, and details of the methods and systems disclosed herein without departing from the spirit and scope of the overall disclosure.

As used herein, unless otherwise specified, the terms “about,” “approximately,” etc., when used in relation to numerical limitations or ranges, mean that the recited limitation or range may vary by up to 10%. By way of non-limiting example, “about 750” can mean as little as 675 or as much as 825, or any value therebetween. When used in relation to ratios or relationships between two or more numerical limitations or ranges, the terms “about,” “approximately,” etc. mean that each of the limitations or ranges may vary by up to 10%; by way of non-limiting example, a statement that two quantities are “approximately equal” can mean that a ratio between the two quantities is as little as 0.9:1.1 or as much as 1.1:0.9 (or any value therebetween), and a statement that a four-way ratio is “about 5:3:1:1” can mean that the first number in the ratio can be any value of at least 4.5 and no more than 5.5, the second number in the ratio can be any value of at least 2.7 and no more than 3.3, and so on.

The embodiments and configurations described herein are neither complete nor exhaustive. As will be appreciated, other embodiments are possible utilizing, alone or in combination, one or more of the features set forth above or described in detail below.

### BRIEF DESCRIPTION OF THE DRAWINGS

FIGS. 1A, 1B, 1C, and 1D are graphs of the average thickness, tensile modulus, strain at break, and tensile strength, respectively, both parallel and perpendicular to the extrusion direction, of fungal-based textiles made by extrusion processes at varying solids contents, according to embodiments of the present disclosure.

FIGS. 2A, 2B, 2C, and 2D are graphs of the average thickness, tensile modulus, strain at break, and tensile strength, respectively, both parallel and perpendicular to the extrusion direction, of fungal biomass/polyvinyl alcohol composite textiles made by extrusion processes at varying solids contents, according to embodiments of the present disclosure.

FIG. 3A is a photograph of a 50 wt % dispersion of spray-dried fungal biomass in epoxidized linseed oil, according to embodiments of the present disclosure.

FIG. 3B is a photograph of a 10 wt % dispersion of spray-dried fungal biomass in an ethylene-vinyl acetate (EVA) copolymer melt, according to embodiments of the present disclosure.

### DETAILED DESCRIPTION

Unless defined otherwise, all technical and scientific terms used herein have the same meaning as is commonly understood by one of ordinary skill in the art. All patents, applications, published applications, and other publications to which reference is made herein are incorporated by reference in their entirety. If there is a plurality of definitions for a term herein, the definition provided in the Summary prevails unless otherwise stated.

As used herein, unless otherwise specified, the term “aqueous” refers to any mixture or solution that includes water. It is to be expressly understood, therefore, that in an “aqueous” solution as that term is used herein, water may be the only solute, one of two or more solutes, the only solvent, or one of two or more solvents.

As used herein, unless otherwise specified, the term “backing material” or “backing layer” refers to a material, or a layer of material, that is added to a composite article to impart one or more desired mechanical and/or structural characteristics (e.g., improved tensile modulus, improved strain at break, increased rigidity/stiffness, etc.). It is to be expressly understood that a backing material or backing layer, as those terms are used herein, need not be disposed on a “back” (i.e., rear, anterior) aspect of a composite article but may be disposed on any exterior aspect (front, back, top, bottom, sides, etc.) or within an interior of the article (e.g., distributed throughout the article, “sandwiched” between layers of other material(s), etc.).

As used herein, unless otherwise specified, the term “biodegradable” refers to a material that, under a given set of conditions (e.g., the conditions specified in ISO 20136:2017, “Leather—determination of degradability by micro-organisms”), degrades by way of biological action.

As used herein, unless otherwise specified, the term “biomass” refers to a mass of a living or formerly living organism, including fungal, microbial, and plant biomass. Microbial biomass sources can include bacterial, fungal (including higher fungi), and microalgal biomass sources. Plant biomass sources can include any source of plant-based biopolymers such as cellulose, lignin and pectin. By way of non-limiting example, the phrase “filamentous fungal biomass” as used herein refers to a mass of a living or formerly living filamentous fungus.

As used herein, unless otherwise specified, the term “biomat” refers to a cohesive mass of filamentous fungal tissue comprising a network of interwoven hyphae filaments. Biomats as that term is used herein may, but need not, be characterized by one or more of a density of between about 50 and about 200 grams per liter, a solids content of between about 5 wt % and about 20 wt %, and sufficient tensile strength to be lifted substantially intact from the surface of a growth substrate (e.g., a liquid growth medium, a solid fungal composite, or a solid membrane or mesh). Biomats, as that term is used herein, may be produced by any one or more fungal fermentation methods known in the art, such as, by way of non-limiting example, methods described in PCT Application Publications 2020/176758, 2019/099474, and 2018/014004.

As used herein, unless otherwise specified, the term “biomaterial” refers to any tissue or other physical material derived from one or more living or formerly living organisms other than animals (e.g., filamentous fungi, plants, bacteria, algae, yeasts, etc.).

“Biomaterials,” as that term is used herein, may be biomasses or portions thereof, but may also be materials that are directly or indirectly derived from the organism(s) (e.g., extracts, metabolites, oils etc.). Particularly, examples of plant-based biomaterials include cotton, hemp, wool, natural rubber, plant oils, sap (e.g., sap of Hevea brasiliensis), and fruits (e.g., pineapples, oranges, bananas, etc.).

As used herein, unless otherwise specified, the term “colloid” refers to a mixture in which particles of one substance (the “dispersed phase”) are dispersed throughout a volume of a different substance (the “dispersion medium”); for example, the dispersed phase can comprise or consist of microscopic or macroscopic bubbles, particles, etc. Where the dispersed phase and the dispersion medium of a colloid are specifically identified herein, they are separated by a hyphen, with the dispersed phase identified first, e.g., a reference herein to an “oil-water colloid” refers to a colloid in which an oil is the dispersed phase and water is the dispersion medium.

As used herein, unless otherwise specified, the term “colloidal gel” refers to a colloid in which the dispersed phase is a liquid, and the dispersion medium is a solid. Examples of colloidal gels as that term is used herein include but are not limited to agar, hair gel, and opal.

As used herein, unless otherwise specified, the term “composite surface fermentation” refers to any method of fungal fermentation in which a high-purity mass of fungal mycelium is grown on a surface of a fungal/substrate composite, such that a mycelial biomass (as that term is used herein) can readily be isolated or separated from the fungal/substrate composite.

As used herein, unless otherwise specified, the term “degree of swelling” refers to the relative amount of change in the mass of a solid item when the solid is saturated with a liquid. By way of non-limiting example, a solid item that has a mass of 200 g when dry and a mass of 300 g when saturated with water has a degree of swelling in water of 50%, or 0.5. Where the term “degree of swelling” is used herein without explicitly identifying a liquid, the liquid may be assumed to be water.

As used herein, unless otherwise specified, the term “deposit” means to cast, lay down, place, or put a deformable mass of material into a desired spatial configuration, such as by extrusion or other suitable techniques.

As used herein, unless otherwise specified, the term “durable” refers to a material that has at least one of a tear strength of at least about 5 N/mm, a tear force of at least about 5 N, and a tensile strength of at least about 1.5 MPa.

As used herein, unless otherwise specified, the term “emulsion” refers to a colloid in which both the dispersed phase and the dispersion medium are liquids. Examples of emulsions as that term is used herein include but are not limited to lotions, latex, and many biological membranes.

As used herein, unless otherwise specified, the term “foam” refers to a colloid in which the dispersed phase is a gas, and the dispersion medium comprises a liquid. Examples of foams as that term is used herein include but are not limited to shaving cream, soap bubbles, and the “head” of a carbonated or nitrogenated beverage.

As used herein, unless otherwise specified, the terms “hide leather” and “true leather” are interchangeable and each refer to a durable, flexible material created by tanning the hide or skin of an animal.

As used herein, unless otherwise specified, the term “inactivated” refers to a filamentous fungal biomass in which the fungal cells have been rendered nonviable, or enzymes capable of degrading or causing biochemical transformations within the biomass have been deactivated, or both. By extension, the term “inactivation” refers to any method or process by which a filamentous fungal biomass may be inactivated, such as, by way of non-limiting example, boiling, immersion in an organic liquid (e.g., an alcohol, peracetic acid, etc.), irradiation, pressure treatment, rinsing, size reduction, steaming, and temperature cycling.

As used herein, unless otherwise specified, the term “infiltration” refers to the permeation and/or saturation of a solution into a mass of solid, but permeable and/or porous, material, such that the solution or a portion thereof is distributed in the mass of solid material, such as, for example and without limitation, a polymer solution permeating the interstitial spaces in a fungal biomat comprised of mycelia. Without being bound by theory, the infiltration of a fungal mycelial biomass with a solution comprising components such as polymers and plasticizers, results in a biomaterial-based material having such components distributed in the biomass after the solvent is removed by curing. Such a distribution can be substantially uniformly distributed or not uniformly distributed.

As used herein, unless otherwise specified, the term “liquid aerosol” refers to a colloid in which the dispersed phase is a liquid, and the dispersion medium is a gas.

As used herein, unless otherwise specified, the term “loading ratio” refers to a weight ratio of biomass to polymer in a biomaterial-based material composition.

As used herein, unless otherwise specified, the term “mass loss upon soaking” refers to the relative amount of mass lost by a solid item after soaking in a liquid, disregarding the mass of liquid absorbed by the solid item. By way of non-limiting example, a solid item that has a mass of 100 grams when dry and a mass (disregarding the mass of absorbed liquid) of 95 grams after soaking in water has a mass loss upon soaking in water of 5%. Where the term “mass loss upon soaking” is used herein without explicitly identifying a liquid, the liquid may be assumed to be water.

As used herein, unless otherwise specified, the term “mycelial biomass” refers to a biomass of filamentous fungus in which fungal mycelium makes up at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, at least about 95 wt %, at least about 96 wt %, at least about 97 wt %, at least about 98 wt %, at least about 99 wt %, or substantially all of the biomass; the remainder (i.e., the non-mycelial portion) of the mycelial biomass may consist of other fungal tissues (conidia, fruiting bodies, etc.). More specifically, the term “cohesive mycelial biomass,” as used herein and unless otherwise specified, refers to a mycelial biomass that is produced by a fermentation process other than a submerged fermentation process and that has sufficient tensile strength and structural integrity to be picked up and moved by hand without disintegrating or tearing. Cohesive mycelial biomasses, as that term is used herein, can be produced by any one or more fungal fermentation methods (e.g., liquid surface fermentation, fermentation on the surface of a membrane or mesh scaffold, etc.) in which the filamentous fungus grows in such a way as to form a mass of interwoven mycelia, e.g., methods in which the fungal mycelium/a is/are grown in air or a controlled atmosphere out of a growth medium or feedstock; non-limiting examples of such methods are described in PCT

Application Publications 2020/176758, 2019/099474, and 2018/014004. Non-cohesive mycelial biomasses can be produced by any one or more submerged fermentation methods known in the art, such as, by way of non-limiting example, methods described in U.S. Pat. No. 7,635,492 to Finnigan et al.; most typically, mycelial biomasses recovered submerged fermentation processes are paste-like substances with poor tensile strength and structural integrity and thus are not cohesive mycelial biomasses as that term is used herein.

As used herein, unless otherwise specified, the term “particle” refers to a small, discrete, localized object to which can be ascribed chemical or physical properties such as volume, density, and/or mass. “Particles,” as that term is used herein, may be microscopic or macroscopic; may be in the gas (e.g., air bubbles), liquid (e.g., droplets of the dispersed phase in an emulsion), or solid (e.g., granules of a powder) phase; and may take any of a variety of shapes (e.g., spheres, oblate spheroids, fibers, tubes, rods, etc.). Particles in the solid phase may be referred to herein as “particulates” or “particulate matter.”

As used herein, unless otherwise specified, the terms “phenomenological gel” and “practical gel” are interchangeable and each refer to a material that (1) is readily deformable (i.e., soft) but otherwise behaves substantially as a solid (i.e., has an elastic modulus substantially greater than its dynamic or loss modulus and thus does not readily flow), and (2) comprises two or more components, one of which is a liquid present in substantial quantity (often, but not always, at least about 50 vol % or wt %). Phenomenological or practical gels are typically, but not necessarily formed, by a three-dimensional network of particles, fibers, or polymer chains, which may be covalently or non-covalently bonded (e.g., via electrostatic interactions, hydrogen bonds, van der Waals forces, hydrophobic interactions, etc.). Examples of phenomenological and/or practical gels as those terms are used herein include but are not limited to hydrocolloids (e.g., agar), K-carrageenan, modified starches, ionically crosslinked pectins or alginates, and three-dimensional networks of silica nanoparticles (e.g., fumed silica).

As used herein, unless otherwise specified, the term “sheet” refers to a layer of solid material having a generally flat or planar shape and a high ratio of surface area to thickness.

As used herein, unless otherwise specified, the term “sol” refers to a colloid in which the dispersed phase is a solid and the dispersion medium is a liquid. Examples of sols as that term is used herein include but are not limited to blood, mud, paint, and pigmented ink.

As used herein, unless otherwise specified, the term “solid aerosol” refers to a colloid in which the dispersed phase is a solid and the dispersion medium is a gas. Examples of solid aerosols as that term is used herein include smoke, ice clouds, and atmospheric particulates.

As used herein, unless otherwise specified, the term “solid foam” refers to a colloid in which the dispersed phase is a gas, and the dispersion medium is a solid. Examples of solid foams as that term is used herein include but are not limited to aerogel, pumice, and styrofoam.

As used herein, unless otherwise specified, the term “solid sol” refers to a colloid in which both the dispersed phase and the dispersion medium are solids. Examples of solid sols as that term is used herein include cranberry glass.

As used herein, unless otherwise specified, the term “tannin” refers generally to any molecule that forms strong bonds with protein structures, and more particularly to a molecule that, when applied to hide leather, bonds strongly to protein moieties within the collagen structures of the skin to improve the strength and degradation resistance of the leather. The most commonly used types of tannins are vegetable tannins, i.e., tannins extracted from trees and plants, and chromium tannins such as chromium (III) sulfate. Other examples of tannins as that term is used herein include modified naturally derived polymers, biopolymers, and salts of metals other than chromium, e.g., aluminum silicate (sodium aluminum silicate, potassium aluminum silicate, etc.).

As used herein, unless otherwise specified, the term “topcoat” refers to a material, or a layer of material, that coats at least one external surface of a biomaterial-based material. It is to be expressly understood that a topcoat, as that term is used herein, need not be disposed on a “top” surface of a biomaterial-based material but may be disposed on any exterior surface (front, back, top, bottom, sides, etc.) thereof.

As used herein, unless otherwise specified, the term “water uptake” refers to the degree of swelling of a solid material when the solid material is saturated with water.

The present disclosure provides biomaterial-based materials, e.g., textiles, having advantageous and beneficial properties, and methods for manufacturing biomaterial-based materials by extruding deformable materials comprising one or more biomaterials, such as, in many embodiments, fungal biomass. In general, the methods of the present disclosure comprise extruding a deformable mixture, comprising biomaterial and a liquid fraction, into a desired spatial configuration, and treating the deformable mixture to form the biomaterial-based material.

In extrusion processes according to the present disclosure, the deformable mixture, comprising the biomaterial and the liquid fraction, is fed into the feed section of an extruder. The temperature and screw speed of the extruder are set to ensure a specific or predefined extent of mixing of materials in the extruder barrel. Multiple feeders can be used to tune the relative loading of each component during the extrusion process. A vacuum port may also be used after a mixing section of the screw to remove air bubbles before the deformable material exits the extruder through the extruder die, which can be in an appropriate shape to achieve the desired cross section of the biomaterial-based material and which can be temperature-controlled. After mixing and degassing, the material exits the die at the end of the extruder in any desired shape (often, though by no means exclusively, in the shape of a sheet by being deposited onto a substantially planar surface).

The deformable mixture is then treated to form the biomaterial-based material. In some embodiments, the treating step may comprise removing at least a portion of the liquid fraction from the extruded deformable mixture, heating the extruded deformable mixture, cooling the extruded deformable mixture, causing a chemical reaction in the extruded deformable mixture (e.g., to cause curing or hardening of the extruded deformable mixture), or a combination thereof. In embodiments in which the treating step comprises removing at least a portion of the liquid fraction from the extruded deformable mixture, this may be carried out, by way of non-limiting example, by heating the deformable mixture, applying a negative pressure to the deformable mixture, radiofrequency irradiation of the deformable mixture, microwave irradiation of the deformable mixture, air drying the deformable mixture, or a combination thereof; in some embodiments, a rate at which the at least a portion of the liquid fraction is removed may be controlled, optimized, selected, and/or tuned to provide a preselected porosity to the biomaterial-based material.

### Properties of Biomaterials and Deformable Extrusion Materials

In the practice of the methods according to the present disclosure, a deformable mixture, comprising one or more biomaterials (e.g., fungal biomass) and a liquid fraction, is extruded into a desired spatial configuration for a biomaterial-based material, such that the biomaterial-based material has the desired spatial configuration after the deformable mixture is treated to form the biomaterial-based material. In many embodiments, this deformable mixture comprises particles of fungal biomass dispersed throughout a dispersion medium comprising the liquid fraction, and it is to be expressly understood, as further described throughout this disclosure, that such fungal particles may be derived from any filamentous fungal organism produced by any known fungal fermentation method (e.g., any one or more of the methods described in U.S. Pat. No. 7,635,492 and/or PCT Application Publications 2018/014004, 2019/099474, and/or 2020/176758). In some embodiments, at least a portion of these filamentous fungal particles may be formed by size-reducing a cohesive fungal biomass (e.g., a biomat of a filamentous fungus produced by a surface fermentation process) by any suitable method, which may, by way of non-limiting example, comprise being processed (e.g., in a blender, food processor, jet mill, or similar size-reducing device), compressed (e.g., by moving jaws, rolls, gyratory cones, or similar compression device), impacted (e.g., by hammer, high-speed jet of material, rollers, or similar impact device), dried (e.g., by spray-drying, flash-drying, supercritical drying, freeze-drying, etc.), and combinations thereof, and the like; this size reduction process may be carried out in any suitable device for any suitable length of time, and the size reduction process may disrupt or destroy at least a portion of a cohesive interconnected or interwoven mycelial network of the fungal biomass. Additionally or alternatively, at least a portion of the filamentous fungal particles may be derived from a biomass that does not require size reduction, such as a fungal paste produced by a submerged fermentation process.

Often, the deformable mixture may be a substantially viscoplastic material that holds its shape (i.e., remains substantially rigid) at low stresses but can be made to deform and/or flow when a stress in excess of the material's yield stress is applied. Materials of this kind are known in the art as Bingham plastics.

It is to be expressly understood that, in any embodiments in which the biomaterial comprises filamentous fungal biomass, any one or more filamentous fungi may suitably be used to form fungal-based materials according to the present disclosure, including but not limited to one or more filamentous fungi belonging to a phylum selected from the group consisting of Ascomycota and Basidiomycota; one or more filamentous fungi belonging to an order selected from the group consisting of Ustilaginales, Sordariales, Russulales, Agaricales, Pezizales, Polyporales, and Hypocreales; one or more filamentous fungi belonging to a family selected from the group consisting of Ustilaginaceae, Sordariaceae, Schizophyllaceae, Hericiaceae, Polyporaceae, Grifolaceae, Lyophyllaceae, Strophariaceae, Lycoperdaceae, Agaricaceae, Pleurotaceae, Physalacriaceae, Omphalotaceae, Tuberaceae, Morchellaceae, Sparassidaceae, Nectriaceae, Ganodermataceae, and Cordycipitaceae; one or more filamentous fungi belonging to a genus selected from the group consisting of Agaricus, Calocybe, Calvatia, Cordyceps, Disciotis, Fomes, Fusarium, Ganoderma, Grifola, Hericulum, Hypholoma, Hypsizygus, Lentinula, Monascus, Morchella, Neurospora, Pholiota, Pleurotus, Polyporous, Schizophyllum, Sparassis, Stropharia, Trametes, Tuber, Ustilago; one or more filamentous fungi belonging to a species selected from the group consisting of Ustilago esculenta, Hericulum erinaceus, Polyporous squamosus, Grifola fondosa, Hypsizygus marmoreus, Hypsizygus ulmarius, Calocybe gambosa, Pholiota nameko, Calvatia gigantea, Agaricus bisporus, Stropharia rugosoannulata, Hypholoma lateritium, Pleurotus eryngii, Pleurotus ostreatus, Pleurotus ostreatus var. columbinus, Tuber borchii, Morchella esculenta, Morchella conica, Morchella importuna, N. crassa, Sparassis crispa, Fusarium venenatum, Fusarium strain flavolapis (ATCC Accession Deposit No. PTA-10698), Disciotis venosa, and Cordyceps militaris; and/or one or more saprobic fungi that derive sustenance from lignin and cellulose-rich sources. The fungal biomass may make up from about 10 wt % to about 85 wt %, or alternatively any amount in any range having a lower bound of any whole number of weight percent from 10 wt % to 85 wt % and an upper bound of any other whole number of weight percent from 10 wt % to 85 wt %, of the deformable mixture and/or the finished biomaterial-based material on a dry basis (i.e., excluding water).

In some embodiments, one or more fillers and/or reinforcing materials, such as natural or synthetic fibers or a combination thereof, may be added to the deformable mixture prior to the treating step to reinforce and provide additional structural integrity to the resulting biomaterial-based material. Non-limiting examples of suitable fillers and/or reinforcing materials include cellulose fibers, cardboard, paper, microfibrillated cellulose, nanofibrillated cellulose, recycled fibers, recycled particles, polymeric fibers, flame retardants, colored pigments, polymeric particles, etc. Most typically, the reinforcing materials, e.g., fibers, may be added to the deformable mixture in an amount of about 2 wt % to about 10 wt %, more preferably about 2.5 wt % to about 9 wt %, and most preferably about 3 wt % to about 8 wt %, or any subrange of any of these ranges, of the deformable mixture on a dry basis (i.e., excluding water).

In some embodiments, one or more plant oils (which may be derived from any part of plant biomass, e.g., leaves, stems, roots, nuts, seeds, etc.) may be added to the deformable mixture prior to the treating step. The plant oil may act in the biomaterial-based material as any one or more of a fragrance, a pest repellent or pesticide, a preservative, a lubricant, a dirt-proofing or dirt resistance agent, a stain-proofing or stain resistance agent, and a waterproofing or water resistance agent. Non-limiting examples of suitable plant oils include cedar oil. Most typically, the plant oil, e.g., cedar oil, may be added to the deformable mixture in an amount of about 0.1 wt % to about 0.5 wt %, more preferably about 0.15 wt % to about 0.35 wt %, and most preferably about 0.2 wt %, or any subrange of any of these ranges, of the deformable mixture on a dry basis (i.e., excluding water).

In some embodiments, one or more salts may be added to the deformable mixture prior to the treating step. The salt may be useful as part of a brine rinse to separate organic contaminants, to promote “salting out” of dyestuff precipitates, to blend with concentrated dyes, to provide a cationic charge to promote absorption of anionic dyes (or vice versa), as an antimicrobial and/or preservative, as a humectant, as a desiccant, etc. Non-limiting examples of suitable salts include sodium chloride, sodium benzoate, and sodium hydroxide. Most typically, the salt, e.g., sodium chloride, may be added to the deformable mixture in an amount of about 0.1 wt % to about 2 wt %, or any subrange thereof, of the deformable mixture on a dry basis (i.e., excluding water).

In some embodiments, one or more dopants (e.g., a thermal dopant, an optical dopant, an electromagnetic dopant, etc.) may be added to the deformable mixture prior to the treating step. A thermal dopant may, but need not, be selected from the group consisting of a ceramic material, a metallic material, a polymeric material, and combinations thereof. Non-limiting examples of suitable thermal dopants include activated charcoal, aluminum oxide, bentonite, diatomaceous earth, ethylene vinyl acetate, lignin, nanosilica, polycaprolactone, polylactic acid, silicone, yttrium oxide, and other ceramic, metallic, and/or polymeric materials. The dopant may alter a selected thermal, optical, and/or electromagnetic characteristic or property of the biomaterial-based material relative to the same characteristic or property in the absence of the dopant. Non-limiting examples of thermal characteristics or properties that may be altered by inclusion of a thermal dopant in the deformable mixture include thermal effusivity, thermal conductivity, and the like heat capacity.

In some embodiments, the deformable mixture prior to the treating step may include, on a dry basis (i.e., excluding water), about 10 wt % to about 85 wt % biomaterial (or any subrange thereof), about 15 wt % to about 50 wt % plasticizer(s) (or any subrange thereof), about 25 wt % to about 75 wt % polymer(s) (or any subrange thereof), and about 2 wt % to about 10 wt % fillers and/or reinforcing materials (e.g., natural or synthetic fibers such as cellulose fibers, microfibrillated cellulose, nanofibrillated cellulose, recycled fibers, recycled particles, polymeric fibers, flame retardants, colored pigments, polymeric particles, etc.). The deformable mixture may further include, on a dry basis about 0.1 wt % to about 0.5 wt % plant oil(s) (e.g., cedar oil) and about 0.1 wt % to about 2 wt % salt(s) (e.g., sodium chloride, sodium benzoate, and/or sodium hydroxide) or any subranges within those ranges. One particular advantage and benefit of the methods of the present disclosure is that the solids content of the deformable mixture may be desirably high (which may in some embodiments allow for the omission of a gelling agent from the deformable mixture), e.g., at least about 10 wt %, at least about 15 wt %, at least about 20 wt %, at least about 25 wt %, at least about 30 wt %, at least about 35 wt %, at least about 40 wt %, at least about 45 wt %, or at least about 50 wt % of the deformable mixture. Additionally or alternatively, the liquid fraction may make up no more than about 90 wt %, no more than about 85 wt %, no more than about 80 wt %, no more than about 75 wt %, no more than about 70 wt %, no more than about 65 wt %, no more than about 60 wt %, no more than about 55 wt %, or no more than about 50 wt % of the deformable mixture.

In some embodiments, the deformable mixture may comprise a gelling agent and/or a non-gelling polysaccharide. A gelling agent may be desirable in certain instances to provide the deformable mixture with certain rheological characteristics, e.g., a desired viscosity and/or yield stress. Non-limiting examples of suitable gelling agents include one or more polymers with a molecular weight of at least about 80,000 daltons, which may, in some embodiments, include one or more polysaccharides, polypeptides, proteins, starches, block copolymers, polyelectrolytes, vegetable gums, hydrocolloids (e.g., ι-karrageenan, κ-carrageenan, λ-carrageenan, agar, starch, modified starch, xanthan, guar gum, locust bean gum, gum arabic, acacia gum, gum karaya, gum tragacanth, alginate, pectin, methyl cellulose, hydroxypropylmethyl cellulose, carboxymethyl cellulose, etc.), and combinations thereof.

In some embodiments, one or more crosslinkers may be added to the deformable mixture prior to the treating step. Crosslinking may be useful to increase solvent resistance (e.g., by reducing swelling or dissolution in solvents), increasing the thermomechanical stability of the material (e.g., by rendering thermoplastics into thermosets), increasing strength and/or elasticity of the material, etc. Particularly, water uptake-resistant or anti-swelling crosslinkers may be useful to decrease the water uptake of the finished biomaterial-based material, i.e., decrease the tendency of the finished biomaterial-based material to absorb water. Non-limiting examples of suitable crosslinkers include polyamideamine-epichlorohydrin (PAE) resins, epoxides, acrylates, and free radical initiators (e.g., organic peroxides). Most typically, crosslinkers, e.g., PAE resin, may be added to the deformable mixture in an amount of about 0.1 wt % to about 10 wt %, or any subrange thereof, of the deformable mixture on a dry basis (i.e., excluding water). Additionally or alternatively, in some embodiments, crosslinking of the deformable mixture may be achieved by electron beam radiation. Additionally or alternatively, the biomaterial-based material may be treated with or otherwise include a waterproofing or water resistance agent, such as lecithin and/or beeswax, in an amount of between about 0.1 wt % and about 40 wt %, or alternatively in any range having a lower bound of any number of tenths of a percent between 0.1 wt % and 40 wt % and an upper bound of any other number of tenths of a percent between 0.1 wt % and 40 wt %. In some embodiments, the water uptake of the finished biomaterial-based material may be no more than about 80%, no more than about 75%, no more than about 70%, no more than about 65%, no more than about 60%, no more than about 55%, no more than about 50%, no more than about 45%, no more than about 40%, no more than about 35%, no more than about 30%, no more than about 25%, no more than about 20%, no more than about 15%, no more than about 10%, no more than about 5%, no more than about 4%, no more than about 3%, no more than about 2%, or no more than about 1%.

The deformable mixture can also include other additives. By way of non-limiting example, the deformable mixture can include suitable amounts of one or more fillers, functionalizing compounds, crosslinkers, polymers, sizing agents, hydrophobing agents, plasticizers, pigments, dyes, antifoaming agents, defoaming agents, flocculants, deflocculants, antimicrobial agents, antistatic agents, UV stabilizers, surface modifiers, foaming agents, blowing agents, and/or flame retardants. The additive may in certain embodiments comprise a surface modifier (e.g., a texture modifier, a slip agent, a non-slip agent, a matting agent, and/or a gloss agent).

In many embodiments, the liquid fraction may be an aqueous liquid fraction, i.e., may comprise water. However, it is to be expressly understood that one particular advantage and benefit of the methods of the present disclosure is that they are effective to extrude non-aqueous deformable mixtures (e.g., deformable mixtures in which the liquid fraction comprises one or more alcohols), and in particular can extrude deformable mixtures in which the liquid fraction itself comprises a material that imparts a desired characteristic, feature, and/or functionality to the finished biomaterial-based material. By way of non-limiting example, the liquid fraction may comprise one or more thermoplastic or non-thermoplastic polymers (e.g., ethylene-vinyl acetate (EVA) and/or ethylene-vinyl alcohol copolymers, polylactic acid, plasticized and/or non-plasticized starch, thermoplastic elastomers such as thermoplastic polyesters (such as the Hytrel series of elastomers produced by Celanese) and particularly segmented polyesters, polyamide block copolymers (such as the PEBAX series of copolymers produced by Arkema), bio-based thermoplastics (such as bio-based polyurethanes, polyesters, and/or polyamides), etc.), either as a melt or dissolved in a nonaqueous solvent, that provide mechanical reinforcement to the finished biomaterial-based material after extrusion and treatment of the deformable mixture. Particularly, the one or more polymers may include at least one thermoplastic elastomer or plasticized thermoplastic, which may provide the finished biomaterial-based material with a desirable tensile modulus and/or improved drapability. In some embodiments in which the deformable mixture comprises a thermoplastic, methods according to the present disclosure may include one or more additional thermoplastic processing steps (e.g., calendering, injection molding, compression molding, etc.) in addition to extrusion.

One type of thermoplastic and/or elastomer that may be particularly desirable for use in biomaterial-based materials according to the present disclosure is polyolefins. Non-limiting examples of suitable thermoplastic polyolefins include polyethylenes (e.g., low-density polyethylene (LDPE), linear low-density polyethylene (LLPE), very-low-density polyethylene (VLDPE), ultra-low-density polyethylene (ULDPE), medium-density polyethylene (MDPE), etc.) polypropylenes (including, e.g., stereo-block polypropylenes), polymethylpentene, polybutene-1, ethylene-octene copolymers, olefin block copolymers, propylene-butane copolymers, natural rubber, and linear or branched polyolefins. Non-limiting examples of suitable polyolefin elastomers include polyisobutene, poly-α-olefins, ethylene propylene rubber (EPR), ethylene propylene diene monomer (EPDM) rubber.

In some embodiments, the deformable mixture may be a foam, i.e., one or more gases (e.g., air, carbon dioxide, nitrogen, gaseous hydrocarbons, etc.) may be incorporated into the fluid mixture, to provide for desired thermal and/or textural characteristics and/or ratio of strength to porosity in the biomaterial-based material. In some embodiments, the deformable mixture may be a sol-gel precursor that, upon treatment of the deformable mixture to form the biomaterial-based material, forms a sol-gel; embodiments of this type may be particularly suitable for forming biomaterial-based materials that include metal and/or ceramic components.

In embodiments in which the biomaterial of the deformable mixture comprises fungal biomass, the particles of fungal biomass in the deformable mixture may be provided by any suitable method. By way of first non-limiting example, a whole or intact biomass or portion thereof (which may, but need not, be inactivated and/or partially or completely dried, e.g., by lyophilization or convective drying) may be size-reduced to form the discrete particles by any one or more suitable size-reduction techniques, such as, by way of non-limiting example, grinding (at either room or cryogenic temperature) and milling. By way of second non-limiting example, “wet” deactivated sheets of biomass may be size-reduced (e.g., using a blade impeller) to a desired fiber length or particle size (e.g., a median fiber length or particle size of less than about 300 μm) and the “cut” sheets may be used in the formulation of the deformable mixture. By way of third non-limiting example, “wet” discrete filaments of mycelium or conidia may be produced by submerged fermentation and used to generate a deformable mixture for extrusion. By way of fourth non-limiting example, discrete mycelial filaments or conidia may be spray-dried into a powder for use in formulation of a deformable mixture. In some embodiments, the spray dried particles are further size reduced by jet milling. In some embodiments, the particles of biomaterial in the deformable mixture may have a multi-modal particle size distribution. By way of fifth non-limiting example, the fungus may be fermented in a water-in-oil emulsion. By way of sixth non-limiting example, the fungus may be fermented as mycelial “shells” on the surface of stabilized air bubbles in a bulk reactor, i.e., by liquid-air interface fermentation, to produce hollow particles.

In some such embodiments, material properties of the composition can be improved by use of fine fungal particles (i.e., fungal particles having a relatively fine particle size). Particularly, the fungal particles may be characterized in that at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or substantially all of the particles of fungal biomass may have a particle size of no more than about 50 μm, no more than about 40 μm, no more than about 30 μm, or no more than about 20 μm.

D_{X} refers to the particle size distribution, wherein X denotes the percentage of particles that are below the indicated size. Thus, D₅₀ means that 50% of the total particles are smaller than the indicated size and D₉₀ means that 90% of the total particles are smaller than this size. In embodiments, the D₅₀ of the particles of fungal biomass may be no more than about 50 μm, no more than about 40 μm, no more than about 30 μm, or no more than about 20 μm; even more particularly, the D₅₀ of the particles of fungal biomass may be about 17 μm to about 23 μm, about 18 μm to about 22 μm, about 19 μm to about 21 μm, and/or about 20 μm. In embodiments, the D₉₀ of the particles of fungal biomass may be about 30 μm to about 40 μm, about 31 μm to about 39 μm, about 32 μm to about 38 μm, about 33 μm to about 37 μm, about 34 μm to about 36 μm, and/or about 35 μm.

In some embodiments, the particle size distribution may be multimodal, i.e., bimodal, trimodal, or consisting of four or more modes.

The deformable mixture may in some embodiments be a colloid. Particularly, the biomaterial may be colloidally dispersed in the liquid fraction.

In some embodiments, the biomaterial-based material can be recycled and re-extruded according to methods of the present disclosure. In some such embodiments, the biomaterial-based material can be combined with 0-99% of a fresh liquid fraction and fresh biomaterial to form a deformable material capable of being extruded. In some such embodiments, the biomaterial-based material can be processed (e.g., by regrinding or re-granulating the material) and re-extruded. In some embodiments, the reground material may be re-compounded with 0-99% of a fresh liquid fraction and fresh biomaterial using a twin screw extruder, and either extruded directly into a desired shape using a die on the twin screw extruder, or pelletized, and then re-extruded into a desired shape with a single screw extruder equipped with a die of the desired shape.

### Extrusion Techniques and Process Parameters

In the practice of the methods of the present disclosure, a deformable mixture comprising one or more biomaterials (e.g., fungal biomass) is extruded, i.e., pushed through a die of a desired cross-section into a desired spatial configuration having the selected cross-section. By extruding the deformable biomaterial-containing mixture, biomaterial-based materials with complex cross-sectional shapes and a high-quality surface finish can be produced, which is often difficult or impossible to achieve by other means.

In some embodiments, the deformable mixture may be extruded onto a substantially planar surface to form a “sheet” of extruded deformable mixture. The substantially planar surface may, but need not, include a textured surface, and/or may comprise biomass particles and be configured to release at least a portion of the biomass particles onto a surface of the extruded deformable mixture such that biomass particles thereby become embedded in the extruded deformable mixture. By way of non-limiting example, it may in some embodiments be desirable to extrude the deformable mixture onto a roll stack containing a textured surface; the textured surface may be applied, by way of non-limiting example, by attaching a release paper with the desired texture onto the roll stack.

Embodiments of the present disclosure may utilize continuous extrusion, allowing for the production of biomaterial-based materials of any arbitrary length, and/or may be semi-continuous, allowing for the production of many identical pieces. Likewise, the deformable mixture, comprising biomaterial and a liquid fraction, may be extruded at ambient and/or room temperature (“cold” extrusion), or above ambient and/or room temperature (“hot” or “warm” extrusion).

Where it is desirable to subject the deformable biomaterial-based mixture to hot or warm extrusion, the extrusion step according to embodiments of the present disclosure may begin by heating the deformable mixture to an extrusion temperature. In all cases, however, the deformable mixture is loaded into an extruder barrel and forced through an extrusion die, for instance by a ram or screw. In some embodiments, the deformable mixture may then be stretched, heat-treated, and/or cold-worked to straighten and/or elongate the material and/or provide the finished biomaterial-based material with one or more desirable mechanical properties.

In some embodiments, the deformable mixture may be subjected to hot or warm extrusion, i.e., the deformable mixture is heated to above ambient/room temperature and subsequently extruded. Heating the deformable mixture prior to extrusion may, in some embodiments, prevent undesirable mechanical and/or rheological phenomena during the extrusion process (e.g., work hardening), decrease the resistance of the mixture to being pushed through the die, and/or result in increased alignment of mycelial filaments (e.g., hyphae) of the biomaterial along a desired axis or in a desired direction. Most typically, the extrusion step in these embodiments may be carried out by a horizontal hydraulic press that exerts a pressure on the deformable mixture. In some embodiments, the interior of the extruder barrel may be lubricated with a suitable lubricant (e.g., oil, graphite, glass powder, etc.).

Additionally or alternatively, in some embodiments, the deformable mixture may be subjected to cold extrusion, i.e., the deformable mixture is extruded at or near ambient/room temperature. Maintaining the deformable mixture at these temperatures may provide any one or more of several benefits, e.g., avoiding oxidation of oxidizable compounds in the mixture, strengthening the mixture via cold working, improving surface finish, and increasing the speed of the extrusion procedure.

In some embodiments, the deformable mixture may be subjected to direct extrusion, i.e., the billet of the deformable mixture is pushed through the die by the ram or screw, with a dummy block placed between the billet and the ram or screw to keep them separated. Additionally or alternatively, the deformable mixture may be subjected to indirect extrusion, i.e., the billet and outer wall of the extruder barrel move while the ram or screw remains stationary (as opposed to vice versa, as is the case in direct extrusion), with the die held in place by a “stem” that is longer than the extruder barrel. Additionally or alternatively, the deformable mixture may be subjected to hydrostatic extrusion, i.e., the billet may be entirely surrounded (except where it is in contact with the die) by a pressurized liquid in a sealed cylinder, where the pressure of the fluid is maintained either by a ram or plunger or by a pump (optionally with a pressure intensifier), to eliminate friction between the billet and the barrel wall and reduce the force needed to push the billet through the die. Regardless of the mechanism used to force the billet through the die, the moving parts of the extrusion device may be driven by any suitable hydraulic or mechanical drive mechanism (e.g., a direct-drive oil press, an accumulator water drive, etc.). The force may be applied against the billet by a single-screw or twin-screw auger, a hydraulically driven ram, or any similar device. In some embodiments, the deformable mixture may be “pelletized,” i.e., formed into discrete pellets, by a twin-screw extrusion device and subsequently processed by molding or a second extrusion step.

In some embodiments, the extrusion step may comprise reactive extrusion, i.e., where a chemical reaction occurs within the deformable mixture during extrusion or shortly before or after extrusion. By way of non-limiting example, fungal biomass in the deformable mixture may be crosslinked during extrusion, or shortly before or after extrusion, by inclusion of a suitable crosslinker (e.g., an acrylate, an epoxy, etc.) in the deformable mixture. Additionally or alternatively, the fungal biomass may be “pre-functionalized” by a crosslinking agent or other similar chemically reactive species prior to being incorporated into the deformable mixture.

In embodiments, the extrusion step may further comprise dual extrusion, co-extrusion, multiple head extrusion, and combinations thereof. In some embodiments, the extrusion step may be a co-extrusion step in which the deformable mixture is extruded together with another material that does not comprise the biomaterial, which may in some embodiments be a coating or backing material. Co-extrusion may be effective to extrude both the deformable mixture and the other material into the same or different spatial configurations; by way of non-limiting example, the deformable mixture and the other material may be extruded as distinct layers, which are then treated to form a multilayer composite. The material that does not comprise biomaterial may in some embodiments comprise a polymer (e.g., one or more of polylactic acid (PLA), a non-PLA thermoplastic such as a polyester or a plasticized starch, a thermoplastic elastomer such as a segmented polyester or a polyamide block copolymer, etc.).

### Properties of Extrusion-Derived Biomaterial-Based Materials

The use of extrusion methods to produce biomaterial-based materials as disclosed herein results in the production of strong, tough biomaterial-based materials that have mechanical, and in many cases aesthetic (i.e., “look and feel”), characteristics comparable to conventional fibrous materials, e.g., leather or other textiles. Particularly, the combination of biomaterial (e.g., fungal biomass) with other components (e.g., plasticizers, crosslinkers, etc.) can give the desired look and feel of conventional fibrous materials. By way of non-limiting example, fungal biomass (and, in some embodiments, fungal mycelium particularly) may be used to impart a desired texture or “hand feel” to the extruded biobased-material; stated slightly differently, the biomass may be effective to reduce the extent to which an extruded biomaterial-based material comprising a thermoplastic subjectively “feels” like a typical plastic to a user (e.g., in terms of smoothness), making articles made from such biomaterial-based materials more appealing in applications in which the tactile properties of the article are important (e.g., automotive trims and seats and similar upholstered items, shoes, apparel, etc.). Non-limiting examples of suitable plasticizers include glycerol, urea, and combinations and mixtures thereof.

In some embodiments, the biomaterial-based materials disclosed herein can have tensile strengths that are suitable for a variety of applications of conventional materials. For example, the biomaterial-based materials can have tensile strengths that are greater than about 3 MPa, greater than about 4 MPa, greater than about 5 MPa, greater than about 6 MPa, greater than about 7 MPa, greater than about 8 MPa, greater than about 9 MPa, greater than about 10 MPa, greater than about 11 MPa, greater than about 12 MPa, greater than about 13 MPa, greater than about 14 MPa, greater than about 15 MPa, greater than about 16 MPa, greater than about 17 MPa, greater than about 18 MPa, greater than about 19 MPa, or greater than about 20 MPa. In other embodiments, the biomaterial-based materials can have tensile strengths from about 3 MPa to about 30 MPa, or in any subrange having a lower bound of any whole number of megapascals from 3 MPa to 30 MPa and an upper bound of any other whole number of megapascals from 3 MPa to 30 MPa.

In some embodiments, the biomaterial-based materials disclosed herein can have a tensile (or flexural) modulus that is suitable for a variety of applications of conventional materials. For example, the materials can have tensile moduli that are greater than about 20 MPa, greater than about 25 MPa, greater than about 30 MPa, greater than about 35 MPa, greater than about 40 MPa, greater than about 45 MPa, greater than about 50 MPa, greater than about 60 MPa, greater than about 70 MPa, greater than about 80 MPa, greater than about 90 MPa, greater than about 100 MPa, greater than about 125 MPa, greater than about 150 MPa, greater than about 175 MPa, or greater than about 200 MPa. In other embodiments, the biomaterial-based materials can have tensile moduli from about 20 MPa to about 200 MPa, or in any range having a lower bound of any whole number of megapascals from 20 MPa to 200 MPa and an upper bound of any other whole number of megapascals from 3 MPa to 30 MPa. Alternatively, in some embodiments, the biomaterial-based materials disclosed herein can have a tensile modulus of no more than about 100 MPa, no more than about 95 MPa, no more than about 90 MPa, no more than about 85 MPa, no more than about 80 MPa, no more than about 75 MPa, no more than about 70 MPa, no more than about 65 MPa, no more than about 60 MPa, no more than about 55 MPa, no more than about 50 MPa, no more than about 45 MPa, no more than about 40 MPa, no more than about 35 MPa, no more than about 30 MPa, no more than about 25 MPa, or no more than about 20 MPa, or alternatively in any range having a lower bound of any whole number of megapascals from 20 MPa to 100 MPa and an upper bound of any other whole number of megapascals from 20 MPa to 100 MPa.

In some embodiments, it may be especially desirable to generate a biomaterial-based material with a low tensile (or flexural) modulus, resulting in a more drapable product, and a low hardness, resulting in a softer feel. In addition, it may be desirable for these materials to have high tensile strength and/or tear strength, and/or to include a high content of carbonaceous material derived from renewable or recycled sources. The biomaterial-based materials of the present disclosure thus overcome a substantial drawback of conventional materials in the art, as this combination of features (i.e., low tensile modulus, low hardness, high tensile strength, high tear strength, and/or high content of renewable carbon) has previously been extremely difficult or impossible to achieve. Incorporation of biomass as a filler material in earlier composite materials has sometimes been employed to increase the overall composition of renewable carbon, but often, these fillers increase the tensile modulus, resulting in a stiffer composite. The biomaterial-based materials of the present disclosure, by contrast, can have both a low tensile modulus and a high content (e.g., at least about 50 wt %, at least about 60 wt %, at least about 70 wt %, at least about 80 wt %, or at least about 90 wt %) of recycled/renewable carbon, while also maintaining adequate tensile strength (e.g., at least about 5 MPa) and elongation at break (e.g., at least about 50%); without wishing to be bound by any particular theory, the present inventors hypothesize that this combination of advantageous features is achieved by the use of formulations that comprise a combination of particulate biomaterial with one or more thermoplastics having low glass transition temperatures. Such formulations can readily be formed into a desired spatial configuration by extrusion through a die and/or into a mold or cavity. In many embodiments, these composites can be formed into sheets by combining the components in the extruder and co-extruding the combined components.

In some embodiments, the extruding step may comprise two or more separate extruding substeps to provide a layered structure to the resulting biomaterial-based material. Even more particularly, some embodiments of the method may include a first extruding substep in which a first layer of the deformable mixture is cast into a desired spatial orientation; a reinforcing substep in which a layer of a reinforcing material (e.g., natural and/or synthetic fibers, a membrane, a mesh scaffold, etc.) is placed atop the first extruded layer of the deformable mixture; and a second extruding substep in which a second layer of the deformable mixture is cast into a desired spatial orientation atop the layer of reinforcing material. This sequence of steps may be repeated any number of times to create a “sandwiched” material (i.e., with a first layer of reinforcing material between first and second layers of deformable mixture, a second layer of reinforcing material between second and third layers of deformable mixture, etc.). In some, but by no means all, embodiments, a previously extruded layer of the deformable mixture may be subjected to treatment of its deformable mixture before a subsequent layer of the deformable mixture is extruded, while in other embodiments the multiple extruded layers may be subjected to treatment simultaneously. Advantageously, this process of placing layers of reinforcing material between extruded layers of the deformable mixture may allow the reinforcing material to be “embedded” or “hidden” within the finished biomaterial-based material, such that they provide structural integrity to the biomaterial-based material without being visible to an observer (e.g., a consumer purchasing an item of clothing or other article made from the biomaterial-based material) and/or may eliminate the need for adhesives or glues for bonding the biomaterial layers to the reinforcing material layers. In further embodiments, more particularly, some embodiments of the method may include a first reinforcing substep in which a layer of a reinforcing material (e.g., natural and/or synthetic fibers, a membrane, a mesh scaffold, etc.) is placed in a position and a second extruding substep in which a layer of the deformable mixture is extruded into a desired spatial orientation atop the layer of reinforcing material. In all such embodiments, the reinforcing material can be placed in position and held under tension for a desired time period. For example, the reinforcing material can be held under tension until the deformable mixture is cast but before, or after, the deformable mixture is treated to form the biomaterial-based material. For example, maintaining the reinforcing material under tension until the deformable mixture is treated can improve the texture or smoothness of the resulting product, such as by reducing or eliminating wrinkles in the final material.

Embodiments of the present disclosure enable the creation of biomaterial-based materials that are resilient to repeated flexing. By way of non-limiting example, the materials of the present disclosure can withstand at least about 5,000, at least about 10,000, at least about 15,000, at least about 20,000, at least about 25,000, at least about 30,000, at least about 35,000, or at least about 40,000 flex cycles in flex cycle testing according to BS EN ISO 5402:2009. In other embodiments, materials of the present disclosure can withstand from about 5,000 to about 1,000,000 flex cycles, or a number of flex cycles in any range having a lower bound of any whole number from 5,000 to 1,000,000 and an upper bound of any other whole number from 5,000 to 1,000,000.

Biomaterial-based materials according to the present disclosure may be manufactured such that they are characterized by a desired strain at break. In some embodiments, by way of non-limiting example, the biomaterial-based materials may be manufactured to have a strain at break of at least about 5 percent, at least about 10 percent, at least about 15 percent, at least about 20 percent, at least about 25 percent, at least about 30 percent, at least about 35 percent, at least about 40 percent, at least about 45 percent, at least about 50 percent, at least about 55 percent, at least about 60 percent, or at least about 65 percent, or of no more than about 70 percent, no more than about 65 percent, no more than about 60 percent, no more than about 55 percent, no more than about 50 percent, no more than about 45 percent, no more than about 40 percent, no more than about 35 percent, no more than about 30 percent, no more than about 25 percent, no more than about 20 percent, no more than about 15 percent, no more than about 10 percent, or alternatively in any range having a lower bound of any whole-number percentage between 1 percent and 70 percent and an upper bound of any other whole-number percentage between 1 percent and 70 percent.

In some embodiments, biomaterial-based materials according to the present disclosure may include a coating material (referred to hereinafter as a “topcoat”), which in some embodiments may comprise or be derived from a polar solvent; this polar solvent may, by way of non-limiting example, be applied to a surface of a biomaterial-based material after deposition of a deformable mixture comprising the biomaterial-based material composition into a desired spatial configuration. In some, but by no means all, cases, the topcoats are polyurethane-based coatings, typically in the form of a polyurethane dispersion (PUD), i.e., a dispersion of polyurethane in a liquid dispersion medium (usually, but not always, water).

In many applications, leather analog materials and other similar textile compositions provided with a topcoat must exhibit an adhesive strength between the topcoat and the biomaterial-based material exceeding a certain threshold when dry and/or when wet, to ensure that the topcoat does not delaminate from the textile. This threshold value is generally at least about 2.0 N per 10 mm. Unlike many previous attempts to provide leather analogs and other similar textiles that incorporate biomaterials, compositions according to the present disclosure can, even when wet, achieve these adhesive strengths, and indeed even greater strengths of at least about 2.2 N per 10 mm, at least about 2.4 N per 10 mm, at least about 2.6 N per 10 mm, at least about 2.8 N per 10 mm, at least about 3.0 N per 10 mm, at least about 3.2 N per 10 mm, at least about 3.4 N per 10 mm, at least about 3.6 N per 10 mm, or at least about 3.8 N per 10 mm.

The coatings/topcoats of the present disclosure may provide the biomaterial-based material with one or more of several advantageous and/or beneficial features in addition to those described elsewhere throughout this disclosure. By way of first non-limiting example, the coatings/topcoats of the present disclosure may improve the abrasion resistance of the material. By way of second nonlimiting example, the coatings/topcoats of the present disclosure may aid in “locking in” plasticizers or other additives, i.e., preventing leaching or separation of these additives from the biomaterial-based material or migration within the biomaterial-based material. By way of third non-limiting example, the coatings/topcoats of the present disclosure may improve the hand feel or other aesthetic qualities (e.g., color, pattern, texture, scent, etc.) of the material, and in particular may provide aesthetic qualities that more closely approximate those of an animal derived material (e.g., hide/true leather) of which the material is an analog. By way of fourth nonlimiting example, the coatings/topcoats of the present disclosure may improve the water repellence capabilities and/or hydrophobicity of the biomaterial-based material. By way of fifth non-limiting example, the coatings/topcoats of the present disclosure may improve the resistance of the biomaterial-based material to damage by ultraviolet light.

Most typically, topcoat material(s) is/are applied to other layers of the biomaterial-based material by spray-coating, but it is to be expressly understood that the topcoat material(s) can also be applied by roll-coating or transfer-coating instead of or in addition to spray-coating. Transfer coatings may be applied by, for example, applying a coating to a transfer paper or transfer film (also known as a release paper or casting paper), and then transferring the coating onto the biomaterial-based material using heat (heat lamination). Sappi casting and release papers (Sappi Ltd. and Sappi North America), including ULTRACAST solvent-free systems, are examples of transfer coating systems that can be employed with the biomaterial-based materials disclosed herein. In some embodiments, the biomaterial-based material, after application of topcoat material(s), may be subjected to plasma, flame, and/or corona treatment, and/or pretreatment with a solvent-borne primer such as Worthen 3296, to enhance adhesion of the topcoat material(s) and facilitate wet-out and adhesion of a topcoating material (e.g., a polyurethane). This pretreatment step may be particularly important where the surface energy of the uncoated biomaterial-based material is highly hydrophobic, as this can create difficulties with wet-out of common leather topcoating materials; flame, plasma, or corona pretreatment can increase the surface energy from about 30 dyne, typical of many biomaterial textile compositions according to the present disclosure, to 50-60 dyne, depending on the watt density of the pretreatment process.

Non-limiting examples of topcoats that may be utilized according to the present disclosure comprise a blend of polyurethanes, acrylics, isocyanates, silicone-based handfeel modifiers, dulling agents, wetting agents, and pigments, and can be spray-coated onto the biomaterial-based material. Before coating, the surface of the material can be chemically modified either via primers and/or electrical or flame treatment. In the case of primers, various commercial products can be used such as Worthen 3213 (halogenation primer), Eastman CP 377W (waterbased APEO free, dispersed chlorinated polyolefin), CP 347W (chlorinated polyolefin), Worthen 3267 (chlorinated polyolefin), or any other chlorinated or non-chlorinated olefin primers. Corona treatment, flame treatment, and/or plasma treatment can also be used as alternatives to these primers. Commercially available polyurethane coatings can be coated onto these chemically modified surfaces. Coating can be done by, for example, transfer films, spray coating, roll coating, or any combination thereof.

In embodiments, the method may further comprise embossing or imprinting a pattern or texture into a surface of the biomaterial-based material. In embodiments, embossing can be performed in-line. For example, the biomaterial-based material may be pressed between textured rollers as it exits the extruder to imprint a pattern. In embodiments, a topcoated or finished biomaterial-based material is embossed. In embodiments, an unfinished or non-topcoated biomaterial-based material is embossed.

In some embodiments, biomaterial-based materials according to the present disclosure may be made into multilayer textiles by extruding the deformable mixture onto a surface of, and/or adhering, laminating, or otherwise affixing the treated deformable mixture to, one or more backing layers of a non-fungal material (e.g., a cotton backing, a nylon backing, a cardboard backing, a paper backing, etc.) and/or a different biomaterial or textile material. In some embodiments, the backing material may be affixed to the biomaterial-based material without the use of an adhesive; by way of non-limiting example, a deformable mixture as disclosed herein may be extruded directly onto a surface of a backing material such that, upon removal of the liquid fraction (or portion thereof) of the deformable mixture, the biomaterial is securely adhered, bonded, or otherwise affixed to the backing material without the use of further adhesives or other additives.

Non-limiting examples of textiles that may be adhered, laminated, or otherwise affixed to biomaterial-based materials according to the present disclosure to form a multilayer textile include an acrylic textile, an alpaca textile, an angora textile, a cashmere textile, a coir textile, a cotton textile, an eisengarn textile, a hemp textile, a jute textile, a Kevlar textile, a linen textile, a microfiber textile, a mohair textile, a nylon textile, an olefin textile, a pashmina textile, a polyester textile, a piña textile, a ramie textile, a rayon textile, a sea silk textile, a silk textile, a sisal textile, a spandex textile, a spider silk textile, a wool textile, and combinations thereof. In some embodiments, the backing material may be chemically pre-treated to provide functional groups on a surface of the backing material that improve the adhesion between the extruded deformable mixture and the backing material. In some embodiments, the backing layer may be a porous or mesh material, which may have a pore size of between about 5 μm and about 25.4 mm, between about 25 μm and about 5.60 mm, between about 0.165 mm and about 2.00 mm, between about 15 μm and about 400 μm, or alternatively in any range having a lower bound of any whole number of microns between 1 μm and 25.4 mm and an upper bound of any other whole number of microns between 1 μm and 25.4 mm. An adhesion force between the layer(s) of biomaterial-based material and the layer(s) of textile material may be at least about 1 N, at least about 2 N, at least about 3 N, at least about 4 N, at least about 5 N, at least about 6 N, at least about 7 N, at least about 8 N, at least about 9 N, at least about 10 N, at least about 11 N, at least about 12 N, at least about 13 N, at least about 14 N, or at least about 15 N (or in any range having an upper bound of any one of these values and a lower bound of any other one of these values), and the multilayer textile may, but need not, be engineered such that a failure mode of adhesion between the layers is either adhesive or cohesive. It is to be expressly understood that multilayer textiles as disclosed herein may include biomaterials in all or less than all of the several layers, and that those layers that include biomaterials may all include the same biomaterial (or combination of biomaterials) or may respectively include different biomaterials (or combinations of biomaterials), e.g., a first layer may include the first biomaterial, a second layer may include a second biomaterial, etc. Multilayer textiles as disclosed herein may have two, three, four, five, six, seven, eight, nine, ten, or more than ten material layers, and may in some embodiments include one or more very thin layers (e.g., having a thickness of no more than about 10 μm).

Biomaterial-based materials according to the present disclosure may be manufactured such that they are characterized by a desired tear strength. In some embodiments, by way of non-limiting example, the biomaterial-based materials may be manufactured to have a tear strength of at least about 5 N/mm, at least about 10 N/mm, at least about 15 N/mm, at least about 20 N/mm, at least about 25 N/mm, at least about 30 N/mm, at least about 35 N/mm, at least about 40 N/mm, at least about 45 N/mm, at least about 50 N/mm, at least about 55 N/mm, at least about 60 N/mm, at least about 65 N/mm, at least about 70 N/mm, at least about 75 N/mm, at least about 80 N/mm, at least about 85 N/mm, at least about 90 N/mm, at least about 95 N/mm, or at least about 100 N/mm, or a tear strength in any range having a lower bound of any whole number of newtons per millimeter from 5 N/mm to 100 N/mm and an upper bound of any other whole number of newtons per millimeter from 5 N/mm to 100 N/mm.

In some embodiments, one or more gases (e.g., air, carbon dioxide, nitrogen, gaseous hydrocarbons, etc.) may be incorporated into biomaterial-based materials according to the present disclosure to produce a “foamed” biomaterial-based material that has a significantly lower mass density than a corresponding “unfoamed” biomaterial-based material. Conversely, in other embodiments, it may be desirable to degas the deformable mixture before, during, or after extrusion to remove any air bubbles and thereby increase the mass density of the biomaterial-based material. In some embodiments, the biomaterial-based material may be “unfoamed” and have a mass density of at least about 1 g/cm³, at least about 1.05 g/cm³, at least about 1.1 g/cm³, at least about 1.15 g/cm³, at least about 1.2 g/cm³, at least about 1.25 g/cm³, at least about 1.3 g/cm³, at least about 1.35 g/cm³, at least about 1.4 g/cm³, at least about 1.45 g/cm³, or at least about 1.5 g/cm³, or in any subrange having a lower bound of any whole number of milligrams per cubic centimeter from 1 g/cm³ to 1.5 g/cm³ and an upper bound of any other whole number of milligrams per cubic centimeter from 1 g/cm³ to 1.5 g/cm³. In other embodiments, the biomaterial-based material may be “foamed” such that the density of the biomaterial-based material is decreased relative to a corresponding “unfoamed” biomaterial-based material by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, e.g., such that the “foamed” biomaterial-based material has a mass density of no more than about 1 g/cm³, no more than about 0.95 g/cm³, no more than about 0.9 g/cm³, no more than about 0.85 g/cm³, no more than about 0.8 g/cm³, no more than about 0.75 g/cm³, no more than about 0.7 g/cm³, no more than about 0.65 g/cm³, no more than about 0.6 g/cm³, no more than about 0.55 g/cm³, no more than about 0.5 g/cm³, no more than about 0.45 g/cm³, no more than about 0.4 g/cm³, no more than about 0.35 g/cm³, no more than about 0.3 g/cm³, no more than about 0.25 g/cm³, no more than about 0.2 g/cm³, no more than about 0.15 g/cm³, no more than about 0.1 g/cm³, or no more than about 0.05 g/cm³. In some embodiments, foamed biomaterial-based materials may also differ from unfoamed biomaterial-based materials in one or more other characteristics or properties, e.g., thermal properties (e.g., thermal effusivity, thermal conductivity, heat capacity, etc.), insulation properties, physical properties (e.g., tensile strength, strain at break, flexibility, etc.), and the like.

Biomaterial-based materials according to the present disclosure may be manufactured such that they are characterized by a desired thickness. In some embodiments, by way of non-limiting example, the biomaterial-based materials may be manufactured to have a thickness of between about 0.1 mm and about 10 cm, or alternatively a thickness in any range having a lower bound of any number of tenths of millimeters between 0.1 mm and 10 cm and an upper bound of any other number of tenths of millimeters between 0.1 mm 10 cm.

Materials of the disclosure can achieve combinations of beneficial properties, such as having both high tensile strength values and high tensile modulus values. For example, such materials can have a tensile strength of greater than about 3 MPa (or any other tensile strength value or within any tensile strength range referenced above) while also having a tensile modulus of greater than about 20 MPa (or any other tensile modulus value or within any tensile modulus range referenced above). Additionally or alternatively, biomaterial-based materials according to the present disclosure may have a biomass content of at least about 10 wt %, at least about 15 wt %, at least about 20 wt %, at least about 25 wt %, at least about 30 wt %, at least about 35 wt %, at least about 40 wt %, at least about 45 wt %, or at least about 50 wt %, while also having one or more of (i) a tensile strength of at least about 1.5 MPa, at least about 3 MPa, or at least about 10 MPa; (ii) a tensile modulus of at least about 20 MPa and/or of no more than about 100 MPa; (iii) a tensile elongation of at least about 40%; (iv) an ability to withstand at least about 5,000 flex cycles in flex cycle testing according to BS EN ISO 5402:2009; (v) a strain at break of at least about 5 percent and/or of no more than about 70 percent; and/or (vi) a tear strength of at least about 5 N/mm.

It is to be expressly understood that the methods and systems disclosed herein are suitable for making biomaterial-based materials from biomaterial-based or particulate non-animal biomasses other than fungal biomasses. Non-limiting examples of such biomaterial-based or particulate, non-animal, non-fungal biomasses include corn stover, sugarcane bagasse, coconut coir, fruit waste, vegetable waste, and spent coffee grounds. The use of certain biomasses, particularly those that may be waste materials from other agricultural or industrial processes, can advantageously produce biomaterial-based materials with desired bulk mechanical, aesthetic, and/or textural properties while mitigating the environmental impact (e.g., carbon emissions) of production of such materials.

Keeping in mind that biomaterials other than fungal biomasses are suitable for use in the methods and systems disclosed herein, in some embodiments, it may be particularly desirable to use fungal biomass as the biomaterial of the deformable mixture. Fungal biomasses, and particularly filamentous fungal biomasses (i.e., biomasses comprising fungal mycelium), may offer any one or more of several advantages. By way of non-limiting example, fungal mycelium may, compared to other biomaterials, more readily form composite materials (i.e., biomaterial-based materials according to the present disclosure) with crosslinkers, plasticizers, and/or other additives, allowing for an increased ability to control, optimize, select, and/or tune various material properties (e.g., softness, protein content, Young's and/or flexural modulus, etc.) by selecting an appropriate plasticizer or other additive (or combination thereof).

Embodiments of the present disclosure are further described by way of the following illustrative and non-limiting Examples.

### Example 1

### Twin-Screw Extrusion of Fungal Biomass Sheets

Sheets of a fungal biomass-containing textile precursor material were formed using a Theysohn twin-screw extruder fitted with an 8″-wide sheet die with adjustable thickness. The twin-screw extruder was equipped with two 21 mm diameter screws and set to operate at 256 rpm, while the barrel and die temperatures were held constant at 82° C.

The throughput of the extruder was maintained at 9.1 pounds per hour by controlling the rate of two separate material feeds to the extruder. Specifically, the first material feed was a feed of K-carrageenan at a constant rate of 1.8 pounds per hour, and the second material feed, which was mixed in downstream of the first material feed, was a feed of a pre-blended aqueous mixture of fungal biomass (in this case, spray-dried particles of Fusarium strain flavolapis produced by a submerged fermentation process), glycerol, urea, and cellulose fibers at a constant rate of 7.3 pounds per hour. Throughout the experiment, the total solids content of the aqueous mixture was systematically increased to produce extruded sheets having total solids contents of 15, 20, 25, and 30 wt %. The compositions of the extruded sheets are given in Table 1 below; note that the second column denotes the total solids (i.e., non-water) content by weight of the sheet material and the five rightmost columns give the weights of the various components of the material on a dry weight basis (i.e., excluding water).

After extrusion, the extruded sheets were frozen before further processing, then subsequently thawed, dried in an oven at 45° C. for 1.5 hours, and conditioned at 25° C. and 50% relative humidity for 24 hours before mechanical testing.

TABLE 1

| Sheet | | | | | | |
|---|---|---|---|---|---|---|
| # | Solids | Biomass | Glycerol | Urea | Cellulose | Carrageenan |
| 1 | 15.0 | 12.6 | 9.3 | 6.0 | 2.2 | 62.9 |
| 2 | 20.0 | 15.0 | 11.0 | 7.5 | 2.6 | 55.9 |
| 3 | 25.0 | 16.9 | 12.4 | 8.4 | 3.0 | 50.4 |
| 4 | 30.0 | 20.6 | 13.5 | 9.2 | 1.1 | 45.8 |

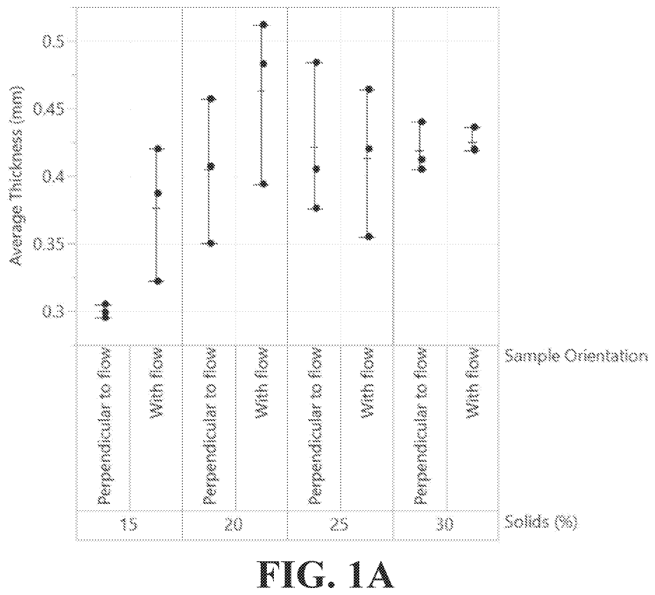

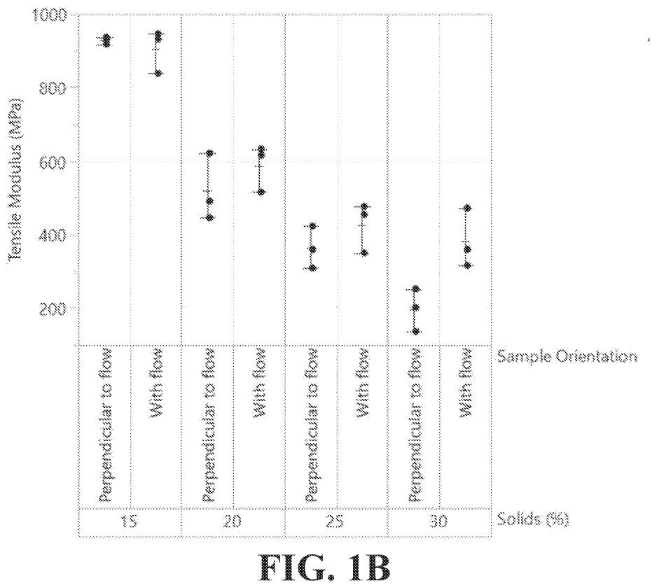

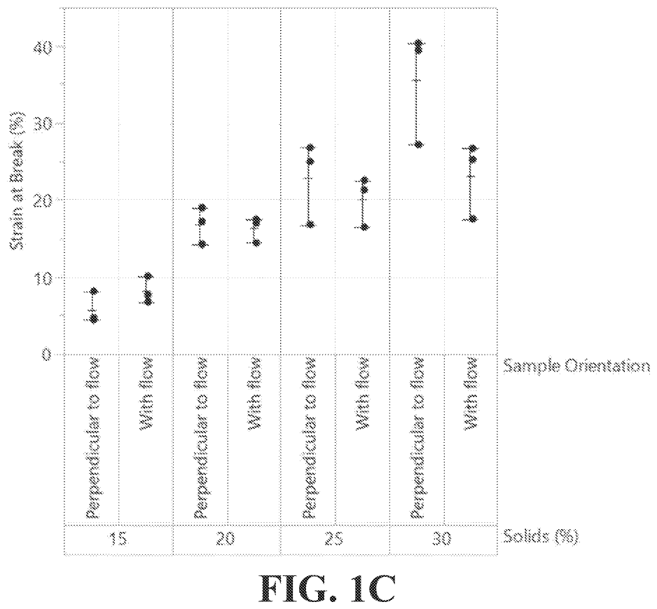

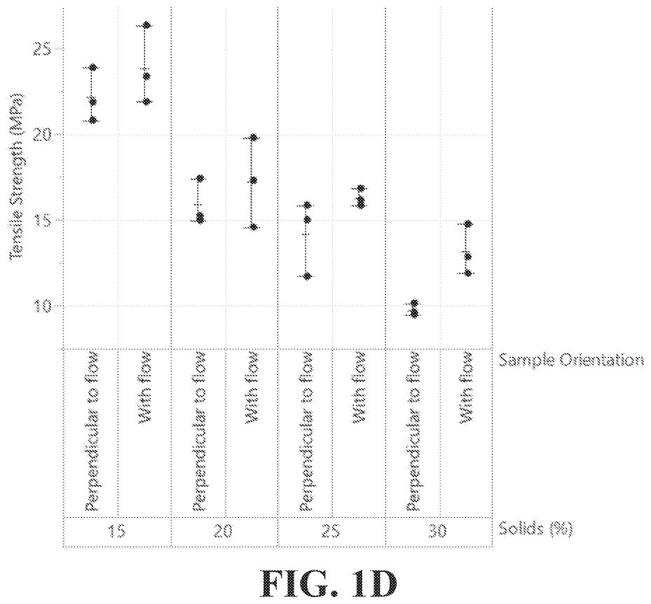

The average thickness, tensile modulus, strain at break, and tensile strength, both parallel and perpendicular to the extrusion direction, are graphed in FIGS. 1A, 1B, 1C, and 1D, respectively.

### Example 2

### Twin-Screw Extrusion of Composite Fungal Biomass/Polyvinyl Alcohol Sheets

Sheets of a composite fungal biomass/polyvinyl alcohol material were formed using a Leistritz twin-screw extruder fitted with a 14″-wide sheet die with adjustable thickness. The twin-screw extruder was equipped with two 27 mm diameter screws and set to operate at 400 rpm.

The throughput of the extruder was maintained at 10 kilograms per hour by controlling the rate of four separate material feeds to the extruder, specifically (1) polyvinyl alcohol powder fed via a loss-in-weight feed device into the extruder feed throat, (2) a blend of agar and fungal biomass (in this case, spray-dried particles of Fusarium strain flavolapis produced by a submerged fermentation process) powder fed into the extruder feed throat, (3) a piston pump-driven feed of water, and (4) a piston pump-driven feed of a mixture of water, glycerol, and urea. The temperature profile of the extruder barrel was optimized for ideal mixing (90° C. in the feeding zone, 105° C. in the mixing zone, 80° C. in a venting zone downstream of the mixing zone, and 70° C. at the die). Throughout the experiment, the total solids content of the aqueous mixture was systematically increased to produce extruded sheets having total solids contents of 35, 40, and 50 wt %; on a dry basis, each sheet consisted of 23 wt % fungal biomass, 25 wt % glycerol, 17 wt % urea, 14 wt % agar, and 21 wt % polyvinyl alcohol.

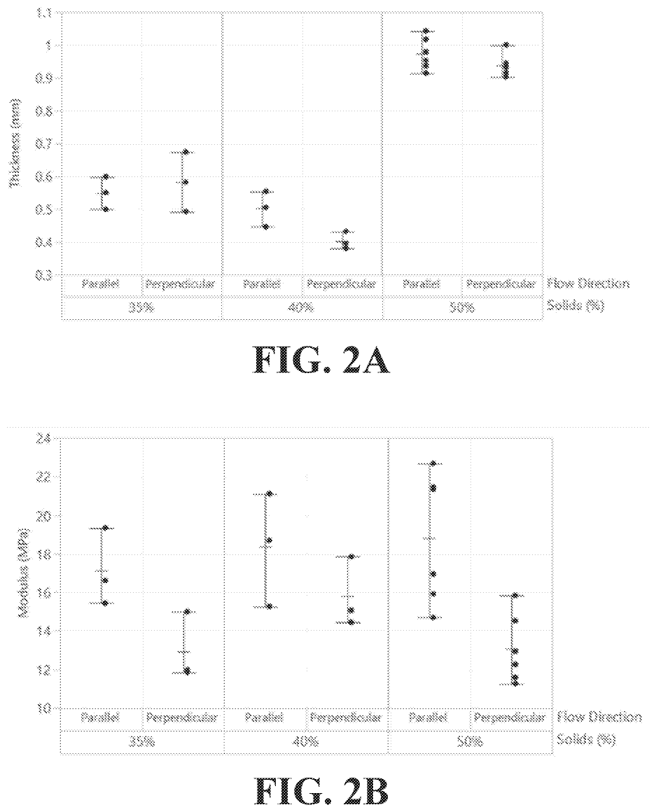

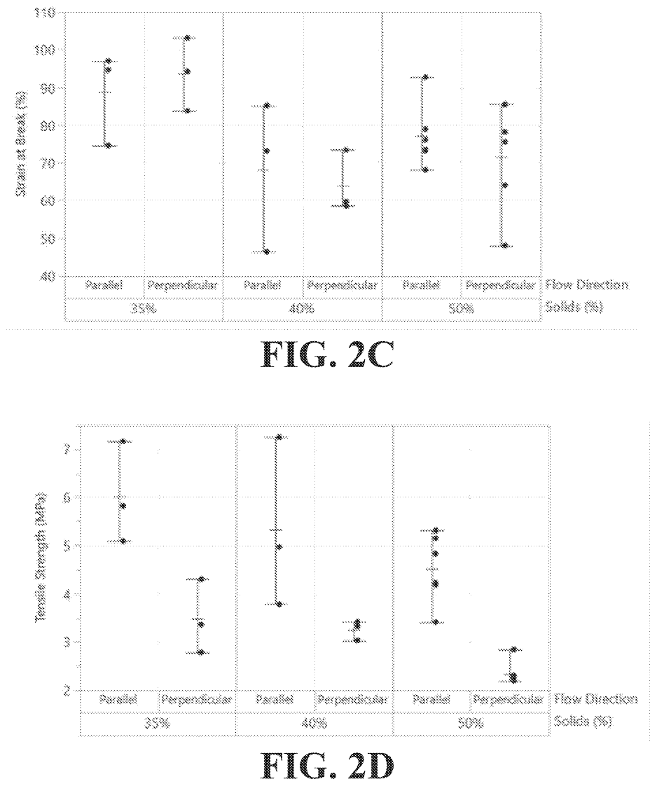

After extrusion, the extruded sheets were dried in an oven at 55° C. for 24 hours and conditioned at 25° C. and 50% relative humidity for 24 hours before mechanical testing. The average thickness, tensile modulus, strain at break, and tensile strength, both parallel and perpendicular to the extrusion direction, are graphed in FIGS. 2A, 2B, 2C, and 2D, respectively.

### Example 3

### Extrusion of Spray-Dried Fungal Biomass-Thermoplastic Composite

Fusarium strain flavolapis biomass was prepared by submerged (stirred-tank) fermentation. After fungal fermentation, steam was injected into the stirred-tank fermenter until a temperature of about 80° C. was achieved in the fermenter to inactivate the fungus. The deactivated biomass was then washed with deionized water and collected as a wet mixture having a solids content of about 25 wt %. This wet mixture was then spray-dried to a solids content of about 98 wt %. The particle size distribution of this spray-dried biomass was determined by sequential sieving and is shown in Table 2 below.

TABLE 2

| Particle size range (μm) | % of particles in range |
|---|---|
| >500 | 0.00 |
| 250-500 | 0.14 |
| 150-250 | 0.28 |
| 75-150 | 52.41 |
| 53-75 | 11.95 |
| 25-53 | 29.19 |
| <25 | 6.03 |

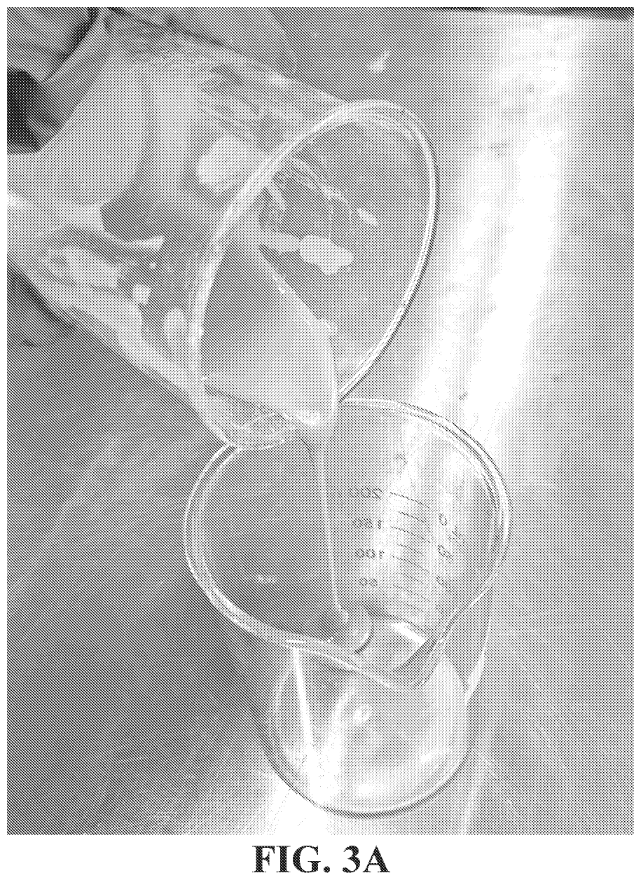

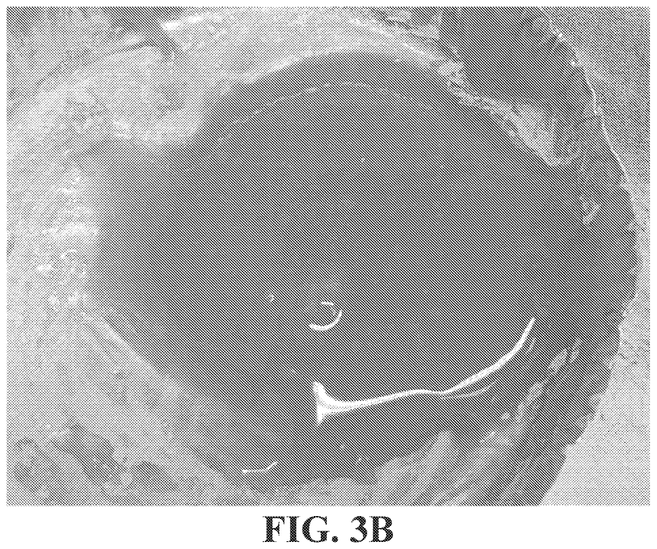

This dried, particulate fungal biomass could be easily dispersed in nonaqueous fluids, such as epoxidized linseed oil (illustrated in FIG. 3A) and an ethylene-vinyl acetate (EVA) copolymer melt (illustrated in FIG. 3B). Specifically, FIG. 3A shows a 50 wt % dispersion of the spray-dried fungal biomass in epoxidized linseed oil, prepared by stirring the fungal biomass in the epoxidized linseed oil in a beaker with a magnetic stir bar for 2 minutes at room temperature, and FIG. 3B shows a 10 wt % dispersion of the spray-dried fungal biomass in the EVA copolymer melt, prepared by hand-mixing the fungal biomass into the pre-melted EVA in an aluminum pan on a hot plate set to 200° C. In both cases, the liquid fraction of the mixture is a colorless, transparent liquid before mixing with the fungal biomass but forms a brown, visually homogeneous (with the exception of some air bubbles) fluid upon mixing with the fungal biomass.

The ease with which spray-dried fungal biomass can be dispersed into these non-aqueous liquids suggests that such mixtures are compatible with both batch mixing processes and continuous mixing and extrusion (e.g., via a twin-screw extruder). In a twin-screw extrusion process, the non-aqueous liquid and the dried fungal biomass powder would be fed to the twin-screw extruder via separate material feeds (which may enter the extruder at the same point, or one downstream of the other, in either order), and the ratio of the two components could be easily controlled utilizing loss-in-weight feeders. Notably, where the liquid fraction is selected to be a polymer melt, the polymer could be fed to the extruder in either solid or liquid form and the extruder could be heated to melt the polymer and/or ensure an appropriate viscosity; by way of non-limiting example, where the liquid fraction is selected to be an EVA copolymer melt, the extruder barrel/twin-screw line could be heated to a temperature of about 200° C. The twin-screw mixer would also apply shear sufficient to ensure adequate dispersion of the fungal particles in the liquid fraction and form the deformable mixture for extrusion, which could then be pelletized or extruded through a slot die to form fungal biomass-reinforced EVA sheets.

### Example 4

Coating formulation recipes obtained from TFL Ledertechnik GmbH were prepared according to Table 3 (obtained from TFL) and used to coat extruded biomaterial-based materials containing fungal biomass (Fusarium flavolapis) combined with either HYTREL 3078 ECO-B thermoplastic polyester elastomer (TPE-E; Celanese) or I'M GREEN SVT 2180 ethylene-vinyl alcohol (EVA) copolymer (Braskem).

TABLE 3

| Component | 1st Coat (g) | 2nd Coat(g) | 3rd Coat(g) |
|---|---|---|---|
| Water | 650 | 150 | 150 |
| RODA Black SH (pigment) | | 100 | 10 |
| EUDERM Driver DE 2 (stir well with water) | 50 | | |
| BAYDERM Pre Bottom APV (PU binder) | 300 | | |
| BAYDERM Bottom PG (PU binder) | | 200 | |
| PRIMAL SCL 371 (general acrylic binder) | | 200 | |
| AQUADERM Finish 85 UD | | 50 | |
| EUDERM X-Grade SF (basecoat filler) | | 150 | |
| EUDERM Matt SN 2 (finishing agent) | | 50 | |
| ACRYSOL RM 2020 E (rheology modifier) | | 10 | |
| AQUADERM XL 50 (crosslinker) | | 50 | |
| RODA Link 5777 (crosslinker) | | 30 | |
| AQUADERM Matt A | | | 550 |
| AQUADERM Gloss A | | | 100 |
| AQUADERM Additive DF (touch modifier) | | | 60 |
| AQUADERM Additive GF (touch modifier) | | | 30 |
| AQUADERM Fluid H (flow control agent) | | | 10 |
| AQUADERM XL 50 | | | 100 |
| RODA Link 5777 | | | 20 |

Each coating layer was sprayed on an outer surface of the extruded fungal biomass/TPE-E or fungal biomass/EVA biomaterial using an HVLP spray gun, at 50 PSI, and heat gun dried as follows.

The first coating layer was applied by spraying four thin layers for a total buildup of approximately 2.2 g/ft2 total weight. The second coating layer was applied by spraying two layers for a total buildup of approximately 2.2 g/ft2 total weight. The third coating layer was applied by spraying two thin layers for a total buildup of approximately 2.2 g/ft2 total weight. The material was cured for approximately 24 hours at room temperature (22° C.-24° C.). After the coatings were fully dry, each of the coated biomaterial-based material had a look and handfeel resembling leather.

The concepts illustratively disclosed herein suitably may be practiced in the absence of any element which is not specifically disclosed herein. It is apparent to those skilled in the art, however, that many changes, variations, modifications, other uses, and applications of the disclosure are possible, and changes, variations, modifications, other uses, and applications which do not depart from the spirit and scope of the disclosure are deemed to be covered by the disclosure.

The foregoing discussion has been presented for purposes of illustration and description. The foregoing is not intended to limit the disclosure to the form or forms disclosed herein. In the foregoing Detailed Description, for example, various features are grouped together in one or more embodiments for the purpose of streamlining the disclosure. The features of the embodiments may be combined in alternate embodiments other than those discussed above. This method of disclosure is not to be interpreted as reflecting an intention that the claims require more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate embodiment.

Moreover, though the present disclosure has included description of one or more embodiments and certain variations and modifications, other variations, combinations, and modifications are within the scope of the disclosure, e.g., as may be within the skill and knowledge of those in the art, after understanding the present disclosure. It is intended to obtain rights which include alternative embodiments to the extent permitted, including alternate, interchangeable, and/or equivalent structures, functions, ranges, or steps to those claimed, regardless of whether such alternate, interchangeable, and/or equivalent structures, functions, ranges, or steps are disclosed herein, and without intending to publicly dedicate any patentable subject matter.

1-87. (canceled)

88. A method for producing a biomaterial-based material, comprising:
a. extruding a deformable mixture comprising a liquid fraction comprising a thermoplastic polymer and a biomaterial into a desired spatial configuration; and
b. treating the extruded deformable mixture to form the biomaterial-based material.

89. The method of claim 88, wherein the biomaterial comprises fungal biomass, and wherein the thermoplastic polymer is selected from ethylene-vinyl acetate, ethylene-vinyl alcohol copolymers, polylactic acid, starch, thermoplastic elastomers, polyamide block copolymers, bio-based thermoplastics, and combinations thereof.

90. The method of claim 89, wherein the thermoplastic polymer is selected from ethylene-vinyl acetate, thermoplastic elastomers, and combinations thereof.

91. The method of claim 89, wherein the thermoplastic polymer is a bio-based thermoplastic.

92. The method of claim 91, wherein the bio-based thermoplastic is selected from polyurethanes, polyesters, polyamides and combinations thereof.

93. The method of claim 88, wherein the biomaterial comprises fungal biomass.

94. The method of claim 88, wherein the biomaterial makes up from about 10 wt % to about 30 wt % of the deformable mixture.

95. The method of claim 88, wherein the extruding step comprises dual extrusion, co-extrusion, and/or multiple head extrusion.

96. The method of claim 88, wherein the extrusion step comprises co-extruding a material that does not comprise the biomaterial and wherein the co-extruded material that comprises the biomaterial has a tensile strength of at least about 3 MPa and/or an elastic modulus of no more than about 70 MPa.

97. The method of claim 96, wherein the co-extruding step comprises co-extruding the deformable mixture with a coating or backing material.

98. The method of claim 96, wherein the co-extruding step comprises extruding a layer of the deformable mixture between first and second layers of the material that does not comprise the biomaterial.

99. The method of claim 96, wherein the material that does not comprise the biomaterial comprises a first polymer.

100. The method of claim 96, wherein the material that does not comprise the biomaterial comprises a thermoplastic elastomer.

101. The method of claim 100, wherein the thermoplastic elastomer is a segmented polyester.

102. The method of claim 100, wherein the thermoplastic elastomer is a polyamide block copolymer.

103. The method of claim 96, wherein at least one of the deformable mixture and the material that does not comprise the biomaterial comprises at least one additive selected from the group consisting of pigments, foaming agents, crosslinkers, plasticizers, polymers other than the first polymer, and biomaterials other than the biomaterial.

104. The method of claim 96, wherein at least one of the following is true:
a. in the extruding step, the deformable mixture is extruded into a first number of layers, wherein the first number of layers is an integer greater than or equal to 1 and less than or equal to 10,000; and
b. in the co-extruding step, the material that does not comprise the biomaterial is extruded into a second number of layers, wherein the second number of layers is an integer greater than or equal to 1 and less than or equal to 10,000.

105. The method of claim 88, wherein the deformable mixture is a colloid in which the biomaterial is dispersed in the liquid fraction.

106. The method of claim 88, wherein the biomaterial-based material is a textile.

107. The method of claim 88, wherein the desired spatial configuration is a sheet, the extruding step comprises extruding the deformable mixture onto a substantially planar surface, and the treating step comprises applying a negative pressure to the deformable mixture, radiofrequency irradiation of the deformable mixture, or microwave irradiation of the deformable mixture.

108. A biomaterial-based material made by the method of claim 88.

109. The biomaterial-based material of claim 108, wherein at least one of the following is true:
a. the biomaterial makes up from about 10 wt % to about 30 wt % of the deformable mixture;
b. the biomaterial makes up at least about 10% by weight of the biomaterial based material;
c. a tensile strength of the biomaterial-based material is at least about 3 MPa; and
d. an elastic modulus of the biomaterial-based material is no more than about 70 MPa.

110. The biomaterial-based material of claim 109, wherein any two of a, b, c, and d are true.
